(19) United States
(12) Patent Application Publication
(10) Pub. No.: US 2005/0272977 A1
(43) Pub. Date: Dec. 8, 2005

(54) Methods and apparatus for performing endoluminal procedures

(75) Inventors: Vahid Saadat, Saratoga, CA (US); Chris Rothe, San Jose, CA (US); Richard C. Ewers, Fullerton, CA (US); Gilbert Madrid, Dana Point, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: USGI Medical Inc., San Clemente, CA (US)

(21) Appl. No.: 11/129,513

(22) Filed: May 13, 2005

Related U.S. Application Data
(63) Continuation-in-part of application No. 10/824,936, filed on Apr. 14, 2004.
(60) Provisional application No. 60/670,426, filed on Apr. 11, 2005.

(51) Int. Cl. A61B 1/04

(52) U.S. Cl. 600/114

(57) ABSTRACT

Methods and apparatus for performing endoluminal procedures are described herein. An endoluminal tissue manipulation assembly is disclosed which provides for a stable endoluminal platform and which also provides for effective triangulation of tools. Such an apparatus may comprise an optionally shape-lockable elongate body defining a longitudinal axis and adapted for endoluminal advancement in a patient body, at least one articulatable visualization lumen disposed near or at a distal region of the elongate body, the at least one articulating visualization lumen being adapted to articulate off-axis relative to a longitudinal axis of the elongate body, and at least one articulatable tool arm member disposed near or at the distal region of the elongate body, the at least one articulatable tool arm member being adapted to articulate off-axis and manipulate a tissue region of interest.

### CROSS-REFERENCES TO RELATED APPLICATIONS

The present application claims the benefit of priority to U.S. Provisional Pat. App. Ser. No. 60/670,426 (Attorney Docket No. 021496-000720US), filed Apr. 11, 2005, and is a continuation-in-part of U.S. patent application Ser. No. 10/824,936 (Attorney Docket No. 021496-000700US), filed Apr. 14, 2004, each of which is incorporated herein by reference in its entirety.

### BACKGROUND OF THE INVENTION

Field of the Invention

The present invention relates to methods and apparatus for performing endoluminal procedures within a body lumen. More particularly, the present invention relates to methods and apparatus for visualizing and/or performing procedures endoluminally within a body lumen utilizing off-axis articulation and/or visualization.

Medical endoscopy entails the insertion of an elongate body into a body lumen, conduit, organ, orifice, passageway, etc. The elongate body typically has a longitudinal or working axis and a distal region, and a visualization element disposed near the distal region in-line with the working axis. The visualization element may comprise an optical fiber that extends through the elongate body, or a video chip having an imaging sensor, the video chip coupled to or including a signal-processing unit that converts signals obtained by the imaging sensor into an image. The elongate body may also include a working lumen to facilitate passage of diagnostic or therapeutic tools therethrough, or for injection of fluids or to draw suction.

The maximum delivery profile for a medical endoscope may be limited by the cross-sectional profile of the body lumen, conduit, organ, orifice, passageway, etc., in which the endoscope is disposed. At the same time, advances in therapeutic endoscopy have led to an increase in the complexity of operations attempted with endoscopes, as well as the complexity of tools advanced through the working lumens of endoscopes. As tool complexity has increased, a need has arisen in the art for endoscopes having relatively small delivery profiles that allow access through small body lumens, but that have relatively large working lumens that enable passage of complex diagnostic or therapeutic tools. Furthermore, as the complexity of operations attempted with endoscopes has increased, there has arisen a need for enhanced visualization platforms, including three-dimensional or stereoscopic visualization platforms.

As with endoscopy, ever more challenging procedures are being conducted utilizing laparoscopic techniques. Due to, among other factors, the profile of instruments necessary to perform these procedures, as well as a need to provide both visualization and therapeutic instruments, laparoscopic procedures commonly require multiple ports to obtain the necessary access. Multiple ports also may be required due to the limited surgical space accessible with current, substantially rigid straight-line laparoscopic instruments.

Moreover, conventional endoscopes and instruments provide generally inadequate platforms to perform complex surgeries within patient bodies. The flexible nature of conventional endoscopes and the structural weakness and functional limitations of the instruments passed through small channels within the endoscopes make vigorous tissue manipulation and organ retraction extremely difficult.

Instruments pushed distally through a retroflexed gastroscope, for example, simply push the unsupported endoscope away from the target tissue. As the instrument is further advanced against the tissue surface, the endoscope is typically flexed or pushed away from the tissue region due to a lack of structural rigidity or stability inherent in conventional endoscopes.

Endoscopic surgery is further limited by the lack of effective triangulation due in part to a 2-dimensional visual field typically provided by an endoscope which limits depth perception within the body lumen. Moreover, conventional endoscopic procedures are generally limited to instruments which allow only for co-axial force exertion along a longitudinal axis of the endoscope. and instruments which have an inability to work outside of the endoscopic axis.

In view of the foregoing, it would be desirable to provide methods and apparatus for performing endoluminal procedures that facilitate introduction of the apparatus into relatively small body lumens, while providing for introduction of at least one relatively large tool, as compared to standard endoscopes or laparoscopes. It also would be desirable to provide methods and apparatus that facilitate single port laparoscopy.

### BRIEF SUMMARY OF THE INVENTION

The endoluminal tissue treatment assembly described herein may comprise, in part, a flexible and elongate body which may utilize a plurality of locking links which enable the elongate body to transition between a flexible state and a rigidized or shape-locked configuration. Details of such a shape-lockable body may be seen in further detail in U.S. Pat. Nos. 6,783,491; 6,790,173; and 6,837,847, each of which is incorporated herein by reference in its entirety.

Additionally, the elongate body may also incorporate additional features that may enable any number of therapeutic procedures to be performed endoluminally. An elongate body may be accordingly sized to be introduced per-orally. However, the elongate body may also be configured in any number of sizes, for instance, for advancement within and for procedures in the lower gastrointestinal tract, such as the colon.

The assembly, in one variation, may have several separate controllable bending sections along its length to enable any number of configurations for the elongate body. For instance, in one variation, elongate body may further comprise a bending section located distal of the elongate body; the bending section may be configured to bend in a controlled manner within a first and/or second plane relative to the elongate body. In yet another variation, the elongate body may further comprise another bending section located distal of the first bending section. In this variation, the bending section may be configured to articulate in multiple planes, e.g., 4-way articulation, relative to the first bending section and elongate body. In a further variation, a third bending section may also be utilized along the length of the device.

In yet another variation, each of the bending sections and the elongate body may be configured to lock or shape-lock its configuration into a rigid set shape once the articulation has been desirably configured. An example of such an apparatus having multiple bending sections which may be selectively rigidized between a flexible configuration and a shape-locked configuration may be seen in further detail in U.S. Pat. Pubs. 2004/0138525 A1; 2004/0138529 A1; 2004/0249367 A1; and 2005/0065397 A1, each of which is incorporated herein by reference in its entirety.

As the bending sections may be articulated in any number of configurations via control wires routed through the elongate body, the assembly may be actively steered to reach all areas of the stomach, including retroflexion to the gastroesophageal junction. The assembly may also be configured to include any number of features such as lumens defined through the elongate body for insufflation, suction, and irrigation similar to conventional endoscopes.

Once a desired position is achieved within a patient body, the elongate body may be locked in place. After insertion and positioning, the distal end of a visualization lumen can be elevated above or off-axis relative to the elongate body to provide off-axis visualization. The off-axis visualization lumen may be configured in any number of variations, e.g., via an articulatable platform or an articulatable body to configure itself from a low-profile delivery configuration to an off-axis deployment configuration. The visualization lumen may define a hollow lumen for the advancement or placement of a conventional endoscope therethrough which is appropriately sized to provide off-axis visualization during a procedure.

Alternatively, various imaging modalities, such as CCD chips and LED lighting may also be positioned within or upon the lumen. In yet another alternative, an imaging chip may be disposed or positioned upon or near the distal end of lumen to provide for wireless transmission of images during advancement of the assembly into a patient and during a procedure. The wireless imager may wirelessly transmit images to a receiving unit located externally to a patient for visualization. Various examples of various articulatable off-axis visualization platforms may be seen in further detail in U.S. patent application Ser. No. 10/824,936 filed Apr. 14, 2004, which is incorporated herein by reference in its entirety.

In addition to the off-axis visualization, an end effector assembly having one or more articulatable tools, e.g., graspers, biopsy graspers, needle knives, snares, etc., may also be disposed or positioned upon or near the distal end of the assembly. The tools may be disposed respectively upon a first and a second articulatable lumen. Each of the articulatable lumens may be individually or simultaneously articulated with respect to bending section and the off-axis lumen and any number of tools may be advanced through the assembly and their respective lumens. During advancement endoluminally within the patient body, tools may be retracted within their respective lumens so as to present an atraumatic distal end to contacted tissue. Alternatively, tools may be affixed upon the distal ends of lumens and atraumatic tips may be provided thereupon to prevent trauma to contacted tissue during endoluminal advancement.

Any number of lumens, articulatable or otherwise, may be utilized as practicable. Examples of articulatable lumens are shown in further detail in U.S. Pat. Pubs. 2004/0138525 A1; 2004/0138529 A1; 2004/0249367 A1; and 2005/0065397 A1, each of which have been incorporated by reference above.

The utilization of off-axis visualization and off-axis tool articulation may thereby enable the effective triangulation of various instruments to permit complex, two-handed tissue manipulations. The endoluminal assembly may accordingly be utilized to facilitate any number of advanced endoluminal procedures, e.g., extended mucosal resection, full-thickness resection of gastric and colonic lesions, and gastric remodeling, among other procedures. Moreover, the endoluminal assembly may be utilized in procedures, e.g., trans-luminal interventions to perform organ resection, anastomosis, gastric bypass or other surgical indications within the peritoneal cavity, etc.

### BRIEF DESCRIPTION OF THE DRAWINGS

FIG. 1 shows an illustrative view of one variation of an endoluminal tissue treatment assembly having a handle, an optionally rigidizable elongate body, and an end effector assembly with articulatable off-axis tool arms and articulatable off-axis visualization.

FIGS. 2A and 2B show illustrative perspective views of a variation of the end effector assembly in a deployed configuration and a low-profile delivery configuration, respectively.

FIG. 3 shows a side view of the end effector assembly of FIGS. 2A and 2B.

FIGS. 4A and 4B illustrate a typical view of the articulatable off-axis tool arms performing a procedure on a tissue region of interest from the perspective of the off-axis visualization lumen.

FIG. 5 illustrates another variation of the off-axis visualization lumen in one deployed configuration.

FIG. 6 shows another variation of the end effector assembly in which the off-axis visualization assembly may be utilized with at least one articulatable off-axis tool arm.

FIG. 7 shows another variation of the end effector assembly in which an inflatable balloon may be utilized for providing an atraumatic surface during low-profile advancement of the end effector.

FIG. 8 shows another variation in which a cap may be utilized at the distal end of the assembly to provide an atraumatic surface for low-profile advancement.

FIG. 9 shows yet another variation of the off-axis visualization lumen in which an articulatable lumen disposed upon a reconfigurable platform may be configured such that visualization of the tissue region of interest directly beneath the imager may be provided.

FIG. 10 shows yet another variation of the off-axis visualization lumen attached to the distal end of the elongate body.

FIG. 11 illustrates an exploded assembly view of one variation for the tool arms.

FIG. 12 illustrates a side view of the tool arms in a deployed configuration.

FIGS. 13A to 13D illustrate possible movements of the articulatable off-axis tool arms relative to the elongate body.

FIG. 14 illustrates the possible longitudinal advancement of at least one tool arm relative to the elongate body.

FIG. 15 illustrates the possible rotational motion of at least one tool arm about its longitudinal axis relative to the elongate body.

FIG. 16 illustrates some of the possible articulation of the tool arms relative to one another.

FIGS. 17A and 17B illustrate one example for advancing an elongate body transesophageally into the stomach for performing a procedure.

FIGS. 18A to 18C illustrate another variation of the elongate body having two adjacent sections which are articulatable relative to each other and which are also optionally rigidizable to retain a desired configuration.

FIGS. 18D and 18E illustrate yet another variation of the elongate body having three adjacent sections which are all articulatable relative to each other and which are also optionally rigidizable to retain a desired configuration.

FIGS. 18F to 18H illustrate an example of a three-sectioned variation of the elongate body being advanced transesophageally into the stomach and articulated to position its distal end near or adjacent to the gastroesophageal junction.

FIG. 18I illustrates another example of FIGS. 18F to 18H in which at least one the bendable sections may be articulated in an opposing direction relative to the remaining two bendable sections to further articulate the elongate body within the stomach.

FIG. 19 shows an end view of one variation of the cross-section of the elongate body providing two lumens for their respective tool arms and a single lumen for the visualization apparatus or endoscope.

FIGS. 20A and 20B show end and side views of an example of an individual link through which the working lumens may be positioned.

FIGS. 21A and 21B show other variations of the cross-section of the elongate body providing two lumens for their respective tool arms, a lumen for visualization, and an auxiliary lumen for an additional instrument to be passed therethrough.

FIG. 21C shows a perspective view of an example for lumen positioning relative to one another for the configuration of FIG. 21A.

FIGS. 22A and 22B show perspective detail views of an example of the handle assembly optionally having a rigidizable elongate body; in a first configuration in FIG. 22A, rigidizing control is actuated or depressed to rigidize or shapelock the elongate body and in a second configuration in FIG. 22B where rigidizing control may be released to place the elongate body in a flexible state.

FIG. 22C shows an end view of the handle of FIG. 22B revealing the open lumen for the passage of tools, instruments, and/or visualization fibers, etc., therethrough.

FIG. 23 shows an exploded perspective view of a sealable or gasketed port assembly which may be attached to the handle for passing tools and/or instruments therethrough while maintaining a seal.

FIGS. 24A and 24B illustrate perspective and partial cross-sectional side views, respectively, of yet another variation of the endoluminal tissue treatment assembly having an endoscope which may be passed through an opening in the elongate body, which is optionally rigidizable, for providing off-axis visualization.

FIGS. 25A and 25B illustrate yet another variation where the articulatable sections of the elongate body may be configured to have different lengths.

FIG. 26 shows another variation in which the articulatable tools may be passed through an opening defined along the elongate body which also has an articulatable distal portion to provide for off-axis visualization.

FIGS. 27A to 27C show yet another variation in which the tool arms may be configured to have predetermined configurations once advanced distally of the elongate body.

FIG. 27D shows yet another variation in which the articulatable tool arms may be freely rotated relative to the elongate body.

FIG. 28 shows yet another variation in which an imaging chip, e.g., a CCD chip, may be disposed upon the end of a guidewire having a predetermined configuration to provide for visualization of the tissue region; the imaging chip may transmit its images via wire through the guidewire or wirelessly to a receiver located externally of a patient body.

FIG. 29 shows yet another variation in which an imaging chip may be disposed upon a pivoting member.

FIG. 30 shows another variation where imaging and/or lighting during a procedure may be provided via imaging capsules and/or LEDs temporarily attached within the patient body and which transmit their images wirelessly to a receiver outside the patient body.

### DETAILED DESCRIPTION OF THE INVENTION

Endoluminal access may be achieved more effectively by utilizing off-axis articulation with an endoluminal tissue manipulation assembly advanced within a body lumen, e.g., advanced endoluminally or laparoscopically within the body lumen. As described herein, off-axis articulating elements may act as reconfigurable platforms from which various tools and/or imagers may be advanced or therapies may be conducted. Once the assembly has been desirably situated within the body, a versatile platform from which to access, manipulate, and visualize a greater portion of the body lumen may be deployed from a device having a relatively small delivery profile.

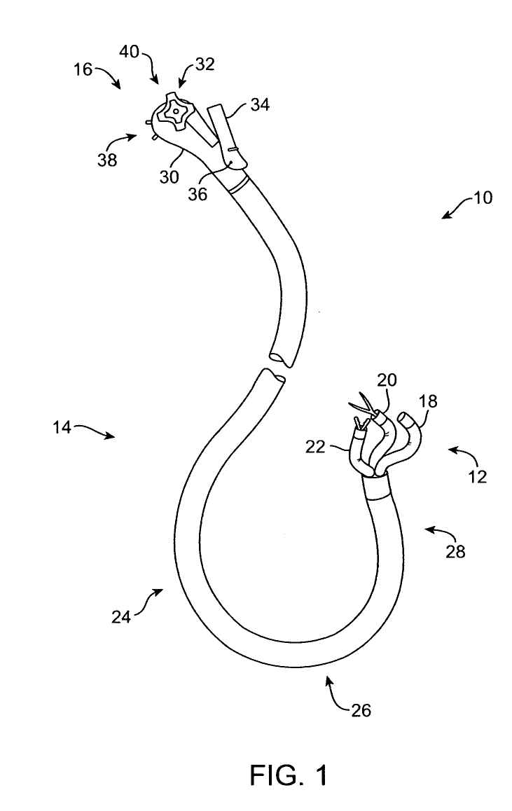

With reference to FIG. 1, the endoluminal tissue manipulation 10 assembly as described herein may comprise, at least in part, a distal end effector assembly 12 disposed or positionable at a distal end of a flexible and elongate body 14. A handle assembly 16 may be connected to a proximal end of the elongate body 14 and include a number of features or controls for articulating and/or manipulating both the elongate body 14 and/or the distal end effector assembly 12.

The elongate body 14 may optionally utilize a plurality of locking or lockable links nested in series along the length of the elongate body 14 which enable the elongate body 14 to transition between a flexible state and a rigidized or shape-locked configuration. Details of such a shape-lockable body may be seen in further detail in U.S. Pat. Nos. 6,783,491; 6,790,173; and 6,837,847, each of which is incorporated herein by reference in its entirety. Alternatively, elongate body 14 may comprise a flexible body which is not rigidizable or shape-lockable but is flexible in the same manner as a conventional endoscopic body, if so desired. Additionally, elongate body 14 may also incorporate additional features that enable any number of therapeutic procedures to be performed endoluminally. Elongate body 14 may be accordingly sized to be introduced per-orally. However, elongate body 14 may also be configured in any number of sizes, for instance, for advancement within and for procedures in the lower gastrointestinal tract, such as the colon.

Elongate body 14, in one variation, may comprise several controllable bending sections along its length to enable any number of configurations for the elongate body 14. Each of these bending sections may be configured to be controllable separately by a user or they may all be configured to be controlled simultaneously via a single controller. Moreover, each of the control sections may be disposed along the length of elongate body 14 in series or they may optionally be separated by non-controllable sections. Moreover, one, several, or all the controllable sections (optionally including the remainder of elongate body 14) may be rigidizable or shape-lockable by the user.

In the example of endoluminal tissue manipulation assembly 10, elongate body may include a first articulatable section 24 located along elongate body 14. This first section 24 may be configured via handle assembly 16 to bend in a controlled manner within a first and/or second plane relative to elongate body 14. In yet another variation, elongate body 14 may further comprise a second articulatable section 26 located distal of first section 24. Second section 26 may be configured to bend or articulate in multiple planes relative to elongate body 14 and first section 24. In yet another variation, elongate body 14 may further comprise a third articulatable section 28 located distal of second section 26 and third section 28 may be configured to articulate in multiple planes as well, e.g., 4-way articulation, relative to first and second sections 24, 26.

As mentioned above, one or each of the articulatable sections 24, 26, 28 and the rest of elongate body 14 may be configured to lock or shape-lock its configuration into a rigid set shape once the articulation has been desirably configured. Detailed examples of such an apparatus having one or multiple articulatable bending sections which may be selectively rigidized between a flexible configuration and a shape-locked configuration may be seen, e.g., in U.S. Pat. Pub. Nos. 2004/0138525 A1, 2004/0138529 A1, 2004/0249367 A1, and 2005/0065397 A1, each of which is incorporated herein by reference in its entirety. Although three articulatable sections are shown and described, this is not intended to be limiting as any number of articulatable sections may be incorporated into elongate body 14 as practicable and as desired.

Handle assembly 16 may be attached to the proximal end of elongate body 14 via a permanent or releasable connection. Handle assembly 16 may generally include a handle grip 30 configured to be grasped comfortably by the user and an optional rigidizing control 34 if the elongate body 14 and if one or more of the articulatable sections are to be rigidizable or shape-lockable. Rigidizing control 34 in this variation is shown as a levered mechanism rotatable about a pivot 36. Depressing control 34 relative to handle 30 may compress the internal links within elongate body 14 to thus rigidize or shape-lock a configuration of the body while releasing control 34 relative to handle 30 may in turn release the internal links to allow the elongate body 14 to be in a flexible state. Further examples of rigidizing the elongate body 14 and/or articulatable sections may again be seen in further detail in U.S. Pat. Pub. Nos. 2004/0138525 A1, 2004/0138529 A1, 2004/0249367 A1, and 2005/0065397 A1, incorporated above by reference. Although the rigidizing control 34 is shown as a lever mechanism, this is merely illustrative and is not intended to be limiting as other mechanisms for rigidizing an elongate body, as generally known, may also be utilized and are intended to be within the scope of this disclosure.

Handle assembly 16 may further include a number of articulation controls 32, as described in further detail below, to control the articulation of one or more articulatable sections 24, 26, 28. Handle 16 may also include one or more ports 38 for use as insufflation and/or irrigation ports, as so desired.

At the distal end of elongate body 14, end effector assembly 12 may be positioned thereupon. In this variation, end effector assembly 12 may include first tissue manipulation arm 20 and second tissue manipulation arm 22, each being independently or simultaneously articulatable and each defining a lumen for the advancement of tools or instruments therethrough. Each of the tools or instruments may be advanced through tool ports 40 located in handle assembly 16 to project from articulatable arms 20, 22 and controlled from handle assembly 16 or proximal to handle assembly 16. Alternatively, various tools or instruments may be attached or connected directly to the distal ends of arms 20, 22 and articulatable from handle assembly 16. At least one of the articulatable arms 20, 22 may be articulatable to reconfigure from a low-profile straightened configuration to a deployed configuration where at least one of the arms 20, 22 is off-axis relative to a longitudinal axis of elongate body 14. Various articulation and off-axis configurations for articulatable arms 20, 22 may be seen in further detail in U.S. Pat. Pub. Nos. 2004/0138525 A1, 2004/0138529 A1, 2004/0249367 A1, and 2005/0065397 A1, incorporated above by reference.

End effector assembly 12 may further include a visualization lumen or platform 18 which may be articulatable into a deployed configuration such that a lumen opening or distal end of visualization lumen or platform 18 is off-axis relative to the longitudinal axis of elongate body 14, as described in further detail below.

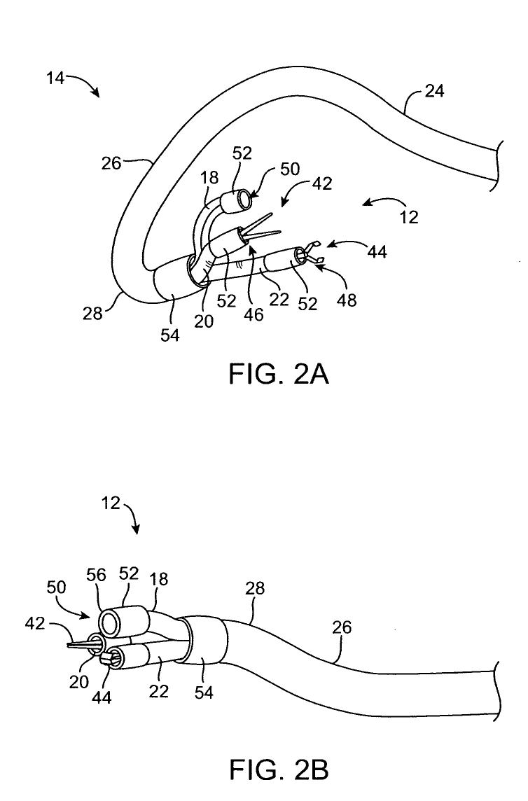

FIGS. 2A and 2B show illustrative perspective views of a variation of the end effector assembly 12 in a deployed configuration and a low-profile delivery configuration, respectively. As seen in FIG. 2A, first and second articulatable arms 20, 22, respectively, may be seen in an off-axis configuration with a first tool 42, e.g., any conventional tool such as a Maryland dissector, Babcock graspers, etc., advanced through first tool lumen 46 within first articulatable arm 20. Likewise, second articulatable arm 22 may have a second tool 44, e.g., any conventional tool such as claw graspers, needle knife, etc., advanced through second tool lumen 48 within second articulatable arm 22. First and second tools 42, 44 may be articulated separately or simultaneously for tissue manipulation and advanced freely distally and proximally through their respective tool lumens 46, 48.

Visualization lumen or platform 18 may also be seen in FIG. 2A articulated into its off-axis configuration relative to elongate body 14. Visualization lumen opening 50 defined at the distal end of visualization platform 18 may be seen articulated into an off-axis configuration which directs visualization opening 50 such that the field-of-view provided therefrom is directly over or upon an area occupied by the articulated tool arms 20, 22 and respective tools 46, 48. Visualization from platform 18 may be provided by any number of different methods and devices. In a first example, visualization may be provided by an endoscope 56 having imaging capabilities advanced through elongate body 14 and through visualization platform 18. Imaging endoscope 56 may be advanced distally to project from lumen opening 50 or it may be positioned within visualization platform 18 such that its distal end is proximal of or flush with lumen opening 50. Alternatively, imaging electronics such as CCD imaging chips or any other number of imaging chips may be positioned within visualization platform 18 to provide images of the field-of-view. These electronic images may be transmitted through wires proximally through elongate body 14 or they may alternatively be transmitted wirelessly to a receiver located externally of the patient body, as described below in further detail.

FIG. 2B shows the end effector assembly 12 in a low-profile configuration for endoluminal advancement through a patient body. An atraumatic distal tip 54 may be provided over the distal end of elongate body 14 and separate atraumatic distal tips 52 may also be provided as well over the distal ends of first and second articulatable tool arms 20, 22.

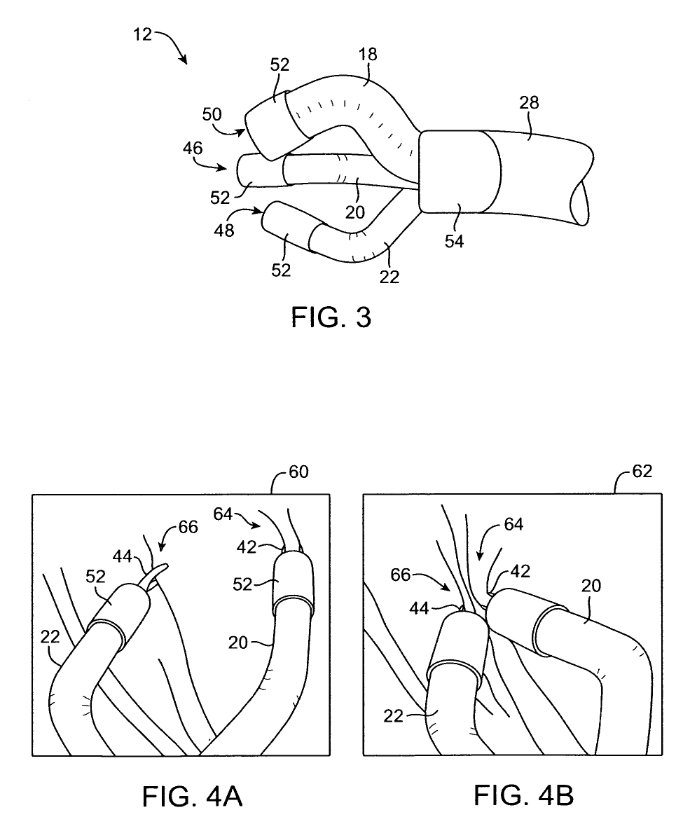

FIG. 3 shows a side view of the end effector assembly 12 of the apparatus of FIG. 2A. As illustrated, first and second tools 42, 44 may be withdrawn into their respective tool lumens 46, 48 during endoluminal advancement of elongate body 14 through the patient and advanced through tool lumens 46, 48 prior to or after articulation of arms 20, 22. Likewise with visualization platform 18, if a visualization endoscope is advanced therethrough, endoscope 56 may be positioned within platform 18 during endoluminal advancement of elongate body 14 or after platform 18 has been articulated.

FIGS. 4A and 4B show an example of the image which an off-axis visualization platform 18 may provide during a tissue manipulation procedure. As seen in FIG. 4A, the visualization image 60 as may be seen on a monitor by the physician during a procedure provides for an off-axis view of the tissue region of interest as well as first and second tools 42, 44 and articulatable arms 20, 22. Such an “overhead” perspective enables the physician to gain an overview of the tissue region of interest during a procedure and facilitates the procedure by further enabling the physician to triangulate the location of the tools 42, 44 with respect to the tissue. Accordingly, manipulation of first tissue region 64 and second tissue region 66 may be readily accomplished by the physician while viewing the tissue region from off-axis platform 18. As seen in the visualization image 62 in FIG. 4B, the tissue regions 64, 66 may be manipulated by articulatable tool arms 20, 22, even when the tissue regions are approximated towards one another; such tissue manipulation and visualization would generally be extremely difficult, if not impossible, using conventional endoscopic devices and tools which are typically limited to straight-line tools and obstructed views typically afforded conventional endoscopes. The utilization of off-axis visualization and off-axis tool articulation may thereby enable the effective triangulation of various instruments to permit complex, two-handed tissue manipulations.

The end effector assembly 12 may accordingly be utilized to facilitate any number of advanced endoluminal procedures, e.g., extended mucosal resection, full-thickness resection of gastric and colonic lesions, and gastric remodeling, among other procedures. Moreover, assembly 10 may be utilized in procedures, e.g., trans-luminal interventions to perform organ resection, anastomosis, gastric bypass or other surgical indications within the peritoneal cavity, etc.

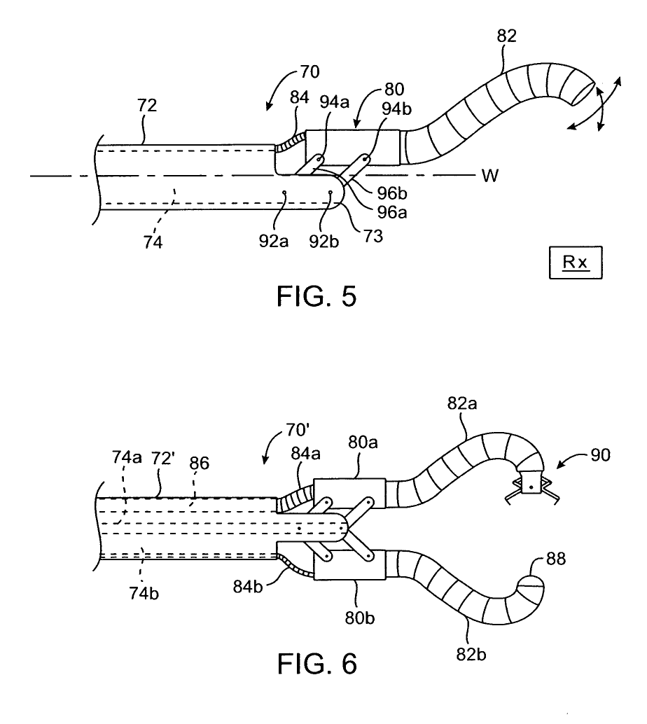

Referring now to FIG. 5, another variation is described wherein the articulating element comprises a steerable shaft. Visualization assembly 70 may generally comprise elongate body 72 having longitudinal axis W, distal region 73 and lumen 74. As mentioned above, elongate body 72 may comprise a rigidizable and/or articulatable body or it may comprise a passively flexible body. Assembly 70 further may further comprise articulating element or platform 80 disposed near distal region 73 of elongate body 72. Platform 80 may be coupled to the elongate body by linkages 96a, 96b rotatably disposed between hinges 92a, 94a and 92b, 94b, respectively. Articulating platform 80 via hinges 92a, 94a and 92b, 94b may allow for lumens or lumen 74 to be unobstructed with the platform 80 articulated away from the openings. Visualization assembly 70 may be seen in further detail in U.S. patent application Ser. No. 10/824,936, which has been incorporated herein above by reference.

Articulating platform 80 may further comprise articulatable visualization lumen 82. Visualization lumen 82 may be passively articulatable or, alternatively, may be actively controllable. Any number of conventional methods may be utilized to articulate the shape and configuration of lumen 82. In FIG. 5, lumen 82 illustratively may, for example, be steerable in any number of directions. In this variation, lumen 82 may be steerable in at least four directions, e.g., via four control wires routed through or along cable 84 and elongate body 72 to a proximal region of assembly 70 for manipulation by a medical practitioner. Cable 84 may also be used to articulate platform 80. The control wires for steerable lumen 82 may be routed through or along body 72 in spaces that would not be usable as working lumens or for tool insertion.

During delivery, articulating platform 80 and steerable lumen 82 are typically aligned with axis W of elongate body 72. Advantageously, the ability to articulate platform 80 off-axis post-delivery allows assembly 70 to have both a large working lumen 74 and a small collapsed delivery profile. Furthermore, steerable platform 82 gives the assembly an off-axis platform with added functionality for performing complex procedures. The steering capability of lumen 82 may be used to steer therapeutic or diagnostic tools, and/or for illumination, visualization, fluid flushing, suction, etc., into better position for conducting such procedures.

Various methods and apparatus for controlling elements used in conjunction with lumen 82 may be routed through cable 84 along with the control wires for lumen 82. For example, when a visualization element is coupled to steerable shaft 82, electrical wires may run through cable 84 for sending and/or receiving signals, power, etc., to/from the visualization element. In such a variation, the visualization element would allow direct visualization during insertion within a body lumen, while providing off-axis visualization and steering, as well as facilitating tool introduction, post-articulation. Alternatively or additionally, when a working lumen is disposed through steerable lumen 82, cable 84 may comprise a lumen for connecting the shaft lumen to a lumen extending through elongate body 72 of assembly 70 through which any number of visualization instruments may be advanced through.

Alternatively or additionally, various imaging modalities, such as CCD chips and LED lighting may also be positioned within or upon lumen 82. In yet another alternative, an imaging chip may be disposed or positioned upon or near the distal end of lumen 82 to provide for wireless transmission of images during advancement of assembly 70 into a patient and during a procedure. The wireless imager may wirelessly transmit images to a receiving unit RX located externally to a patient for visualization.

Referring now to FIG. 6, an alternative variation of assembly 70 is shown comprising multiple articulating elements having steerable shafts. Assembly 70′ may comprise first articulating platform 80a and second articulating platform 80b. Platform 80 may comprise first steerable lumen 82a and second steerable lumen 82b, respectively. Lumens 74a and 74b extend through elongate body 72′ and are exposed upon articulation of platform 80a and 80b, respectively. As will be apparent, a single lumen or more than two lumens alternatively may be provided. Likewise, more than two articulating elements and/or steerable shafts optionally may be provided.

First steerable lumen 82a illustratively is shown with working lumen 86 that extends through the lumen, as well as through cable 84a and elongate body 72′. Exemplary grasper tool 90 is shown advanced through lumen 86. Second steerable lumen 82b illustratively is shown with visualization element 88, as previously described, coupled to an end thereof. Electrical wires, e.g., for powering and transmitting signals to/from the visualization element, may be disposed within cable 84b. As will be apparent, steerable lumens 82 may be provided with additional or alternative capabilities. In the case of visualization element 88 being a wireless imager, electrical wires may be omitted altogether.

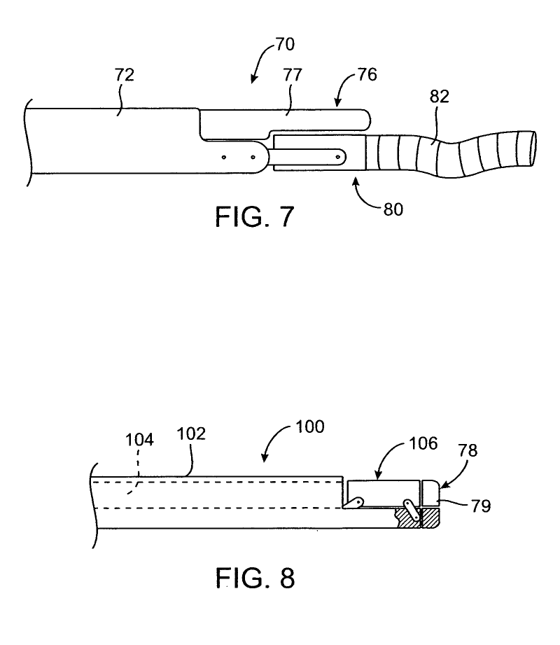

With reference to FIGS. 7 and 8, illustrative embodiments of atraumatic tips for use with the assembly 70 are described. As shown in FIG. 7, assembly 70 is shown with atraumatic tip 76. Tip 76 provides a smooth transition between elongate body 72 and articulating platform 80 with steerable lumen 82. Tip 76 may, for example, comprise an inflatable balloon 77 that may be inflated as shown during insertion and delivery of assembly 70, then deflated prior to articulation of platform 80 and off-axis steering of lumen 82, so as not to block or impede articulation or the distal opening of the lumen 74 post-articulation.

In FIG. 8, assembly 100 may comprise an alternative atraumatic tip 78 having cap 79, which optionally may be fabricated from rubber. Cap 79 may be U-shaped to both provide a smooth transition between elongate body 102 and articulating platform 106 in the delivery configuration, as well as to ensure that the cap does not block or impede lumen 104 post-articulation.

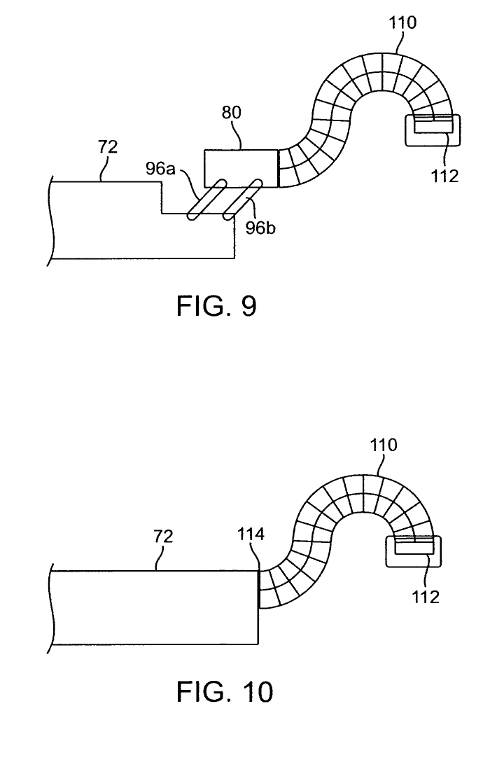

FIGS. 9 and 10 show additional alternative configurations of the articulatable platform and visualization lumen. Articulatable visualization lumen 110 may be manipulated to articulate in an off-axis configuration such that visualization lumen opening 112 is directed to face in a direction which is off-axis relative to a longitudinal axis of elongate body 72 and which is also perpendicular relative to the longitudinal axis. Although visualization lumen 110 may be articulated to face any number of directions, such a configuration may allow for a visualization element positioned within opening 112 to directly face over or upon the tissue region of interest, if so desired.

As shown in FIG. 9, visualization lumen 110 may be positioned upon platform 80 and articulated via linkages 96a, 96b, as described above. Alternatively, visualization lumen 110 may also be directly attached via interface 114 to elongate body 72 and articulated therefrom, also as described above.

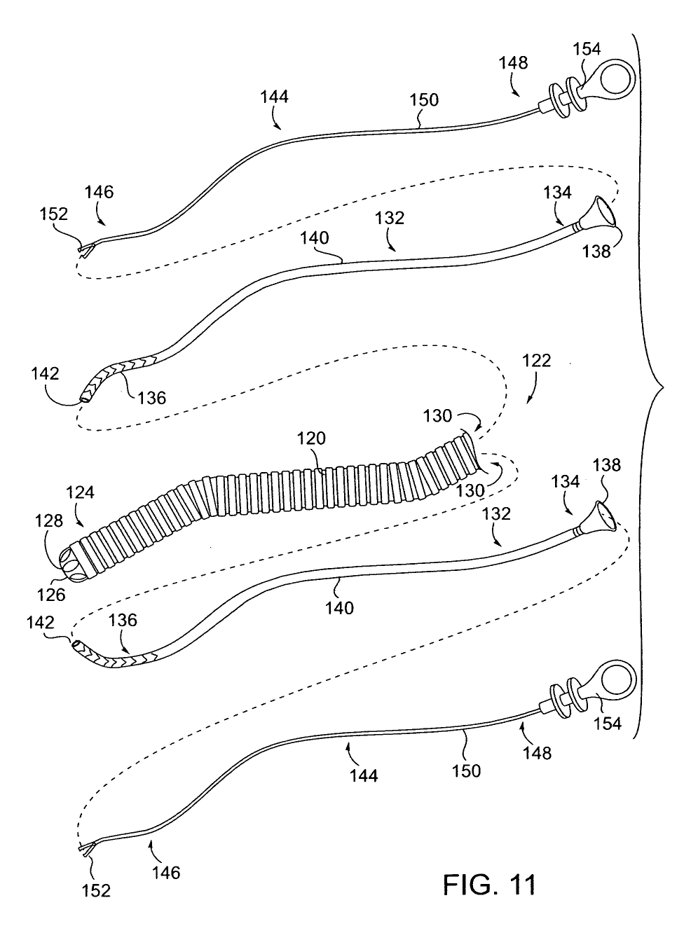

Turning now to the elongate body, FIG. 11 illustrates one variation for assembly of the elongate body 120. Distal end effector assembly 12 has been omitted merely for the sake of clarity from FIG. 11 and following figures. The elongate body 120 may have a single lumen therethrough for a variety of uses, such as for passage of one or more instruments or for the passage of air or fluid, such as for aspiration or suction. Similarly, the elongate body 120 may have more than one lumen passing therethrough, each lumen used for a different function.

Further details of the elongate body construction may be seen in any of the following U.S. Pat. Pubs. 2004/0138525 A1; 2004/0138529 A1; 2004/0249367 A1; and 2005/0065397 A1, each of which is incorporated herein by reference in its entirety.

In some variations, elongate body 120 may include at least one instrument or tool lumen 130, e.g. an arm guide lumen, which extends over or through at least a distal section of the elongate body 120, typically along the majority of the length of the body 120 as shown. Here in FIG. 11, two arm guide lumens 130 are shown, each extending from a position along the shaft 120 near the proximal end 122 to the distal tip 126. In addition, the elongate body 120 includes a visualization lumen 128, which extends through the shaft 120 to the distal tip 126.

In some variations, the assembly also includes at least one tool arm 132, two are shown in FIG. 11, each arm 132 of which is insertable through a separate arm guide lumen 130 as indicated by the dashed lines. Each tool arm 132 has a proximal end 134, a distal end 136 and a shaft 140 therebetween. The distal end 136 optionally is steerable, such as by manipulation of adjacent links as schematically indicated. Such steerability may be controlled by any number of methods, e.g., a steering cuff 138, which is part of the proximal end 134. The shaft 140 is typically flexible or deflectable to allow deflection of the surrounding elongate body shaft 120. Each tool arm 132 may additionally include a tool deployment lumen 142 therethrough.

Elongate body 120 includes at least one tool 144 with two tools 144 shown in FIG. 11. Each tool 144 includes a distal end 146, a proximal end 148 and an elongate shaft 150 therebetween to allow passage through the tool deployment lumen 142 of the tool arm 132, or through lumen 130 of elongate body 120. Each tool 144 has an end effector 152 disposed at the distal end 146 and optionally a handle 154 at the proximal end 148 for manipulation of the end effector 152 from outside the body. The tool 144 is advanced so that the end effector 152 emerges from the distal end 136 of the arm 132, or from distal tip 126 of elongate body 120. As will be apparent, tool 144 optionally may be formed integrally with tool arm 132. Accordingly, rather than utilizing one or more tool arm shafts 140 insertable through elongate body 120, articulatable distal ends 136 may alternatively be connected directly near or at the distal tip 126 of elongate body 120. Additionally, the distal ends of tools 144 may also be connected directly to articulatable distal ends 136.

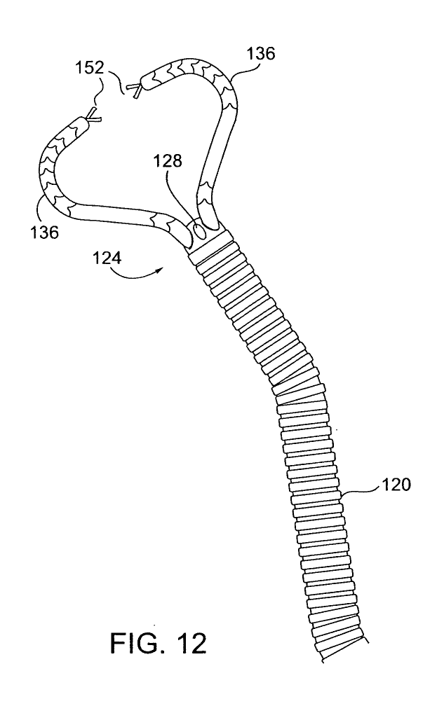

FIG. 12 illustrates the assembly of FIG. 11 in an exemplary assembled arrangement. Here, the tool arms 132 are shown inserted through the arm guide lumens 130 of the elongate body shaft 120. The steerable distal ends 136 of the arms 132 protrude from the distal end 124 of the elongate body 120 and the proximal ends 134 of the arms 132 protrude from the proximal end 122 of the elongate body 120. Additionally, the tools 144 are shown inserted through the tool deployment lumens 142 so that the end effectors 152 extend beyond the steerable distal ends 136 of the arms. Likewise, the proximal ends 148 of the tools 144 with handles 154 may protrude proximally from the assembly. As described above, the articulatable visualization lumen 18 or 110 (omitted from the figure for clarity) may be connected to the distal end of 124 of elongate body 120 at the location of lumen 128. Alternatively, an endoscope used for visualization may be routed directly through lumen 128.

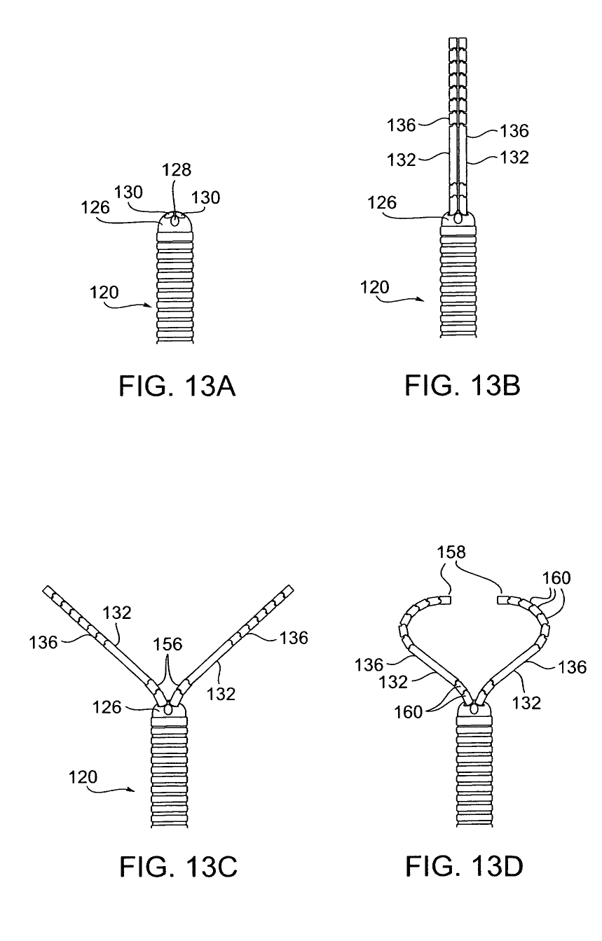

FIGS. 13A to 13D illustrate a series of movements of the steerable distal ends 136 of the tool arms 132. This series serves only as an example, as a multitude of movements may be achieved by the distal ends 136 independently or together. Moreover, articulatable visualization lumen or platform 18 or 110 has been omitted from the illustrations merely for the sake of clarity. FIG. 13A illustrates the distal tip 126 of the elongate body 120. The visualization lumen 128 is shown along with two arm guide lumens 130. FIG. 13B illustrates the advancement of the distal ends 136 of the tool arms 132 through the arm guide lumens 130 so that the arms 132 extend beyond the distal tip 126.

FIGS. 13C and 13D illustrate deflection of the arms 132 to an exemplary arrangement. FIG. 13C illustrates deflection of the arms 132 laterally outward. This may be achieved by curvature in the outward direction near the base 156 of the steerable distal end 136. FIG. 13D illustrates deflection of the tip section 158 of the distal end 136 laterally inward achieved by curvature in the inward direction. When an imager 162 is positioned within the lumen 128, the tip sections 158 of the tool arms 132 and any tools 144 advanced therethrough, will be visible through the imager 162. Additionally, when articulatable visualization lumen 18 or 110 is positioned within or connected to lumen 128, articulation of the visualization lumen into its off-axis configuration will bring tools 132, and in particular the distal ends 136 of tool arms 132 into the field-of-view, as described above. In FIGS. 13C and 13D, deflection of the arms 132 may be achieved with the use of adjacent links 160 in the areas of desired curvature.

Variations of such links 160 and other mechanisms of deflection are described in further detail in U.S. Pat. Pubs. 2004/0138525 A1; 2004/0138529 A1; 2004/0249367 A1; and 2005/0065397 A1, each of which has been incorporated above herein by reference. Further, the deflection shown in FIGS. 13A to 13D are shown to be within a single plane. However, variations include deflection in multiple planes. Likewise, the arms 132 are shown to be deflected simultaneously in FIGS. 13A to 13D, however the arms 132 may be deflected selectively or independently.

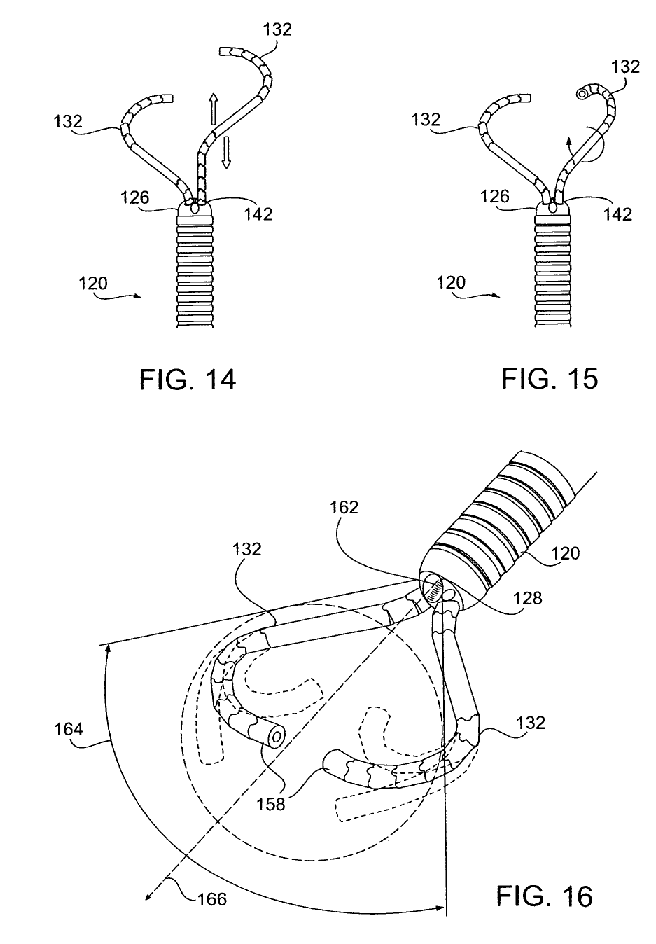

FIGS. 14 to 16 illustrate additional possible movements of the tool arms 132. For example, FIG. 14 illustrates possible axial movement of the tool arms 132. Each tool arm 132 can independently move distally or proximally, such as by sliding within the tool deployment lumen 142, as indicated by the arrows. Such movement may maintain the arms 132 within the same plane, yet allows more diversity of movement and therefore surgical manipulations. FIG. 15 illustrates rotational movement of the tool arms 132. Each tool arm 132 can independently rotate, such as by rotation of the arm 132 within the tool deployment lumen 142, as indicated by circular arrow. Such rotation may move the arm or arms 132 through a variety of planes. By combining axial, lateral and rotational movement, the arms 132, and therefore the tools 144 positioned therethrough (or formed integrally therewith), may be manipulated through a wide variety of positions in one or more planes.

FIG. 16 illustrates further articulation of the tool arms 132. In some variations, the arms 132 may be deflectable to form a predetermined arrangement. Typically, when forming a predetermined arrangement, the arms 132 are steerable up until the formation of the predetermined arrangement wherein the arms 132 are then restricted from further deflection. In other variations, the arms 132 may be deflectable to a variety of positions and are not limited by a predetermined arrangement. Such an example is illustrated in FIG. 16 wherein the arms 132 articulate so that the tip sections 158 curl inwardly. The tip sections 158 may be positioned in front of the lumen 128 and imager 162 for viewing or within the field-of-view provided by the off-axis articulation of visualization lumen 18 or 110 (omitted for clarity). Typically, the tip sections 158 may be positioned on opposite sides of a longitudinal axis 166 of the elongate body 120, wherein for an imager 166 positioned within lumen 128, in one variation, the field-of-view (indicated by arrow 164) may span up to, e.g., approximately 140 degrees.

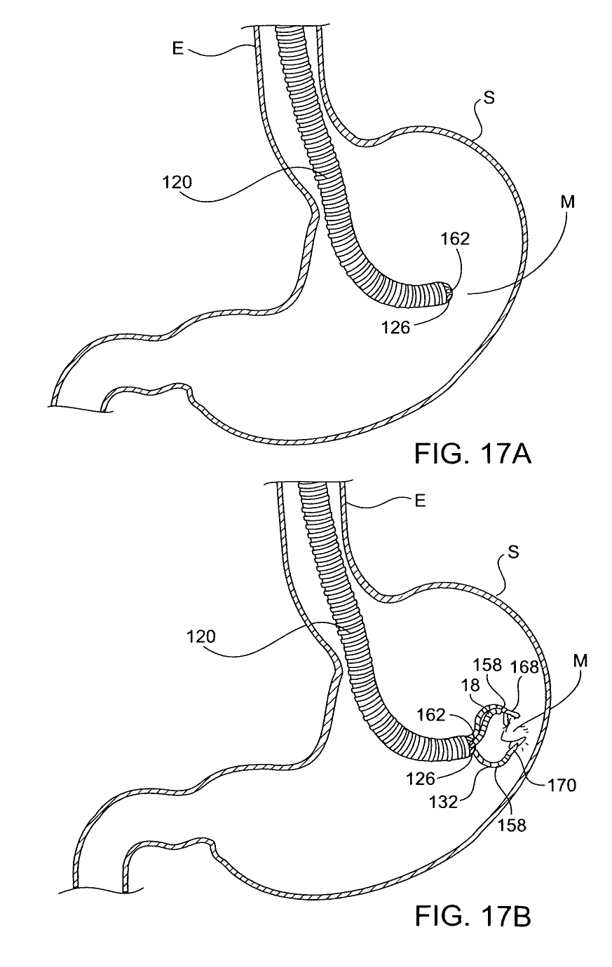

FIGS. 17A and 17B illustrate one example for use of the endoluminal assembly 10. FIG. 17A illustrates advancement of the elongate body 120 through the esophagus E to the stomach S, as shown in FIG. 17A. The elongate body 120 may then be steered to a desired position within the stomach S, and a tissue region of interest M may be visualized by visualization lumen or platform 18, which may be articulated into its off-axis configuration, as shown in FIG. 17B. Tool arms 132 may also be advanced, if not already attached directly to the distal end of elongate body 120, through the elongate body 120 and articulated. As previously described, one or several tools 144 may be advanced through the tool arms 132, or an end effector 152 may be disposed at the distal end of each arm 132. In this example, a grasper 168 is disposed at the distal end of one arm 132 and a cutter 81 is disposed at the distal end of the other arm 132, although any number of tools, e.g., graspers, biopsy graspers, needle knives, snares, etc., may be utilized depending upon the desired procedure to be performed. Moreover, the tools 144 may alternatively be affixed upon the distal ends of tool arms 132 and atraumatic tips may be provided thereupon to prevent trauma to contacted tissue during endoluminal advancement.

It may be appreciated that the systems, methods and devices of the present invention are applicable to diagnostic and surgical procedures in any location within a body, particularly any natural or artificially created body cavity. Such locations may be disposed within the gastrointestinal tract, urology tract, peritoneal cavity, cardiovascular system, respiratory system, trachea, sinus cavity, female reproductive system and spinal canal, to name a few. Access to these locations may be achieved through any body lumen or through solid tissue. For example, the stomach may be accessed through an esophageal or a port access approach, the heart through a port access approach, the rectum through a rectal approach, the uterus through a vaginal approach, the spinal column through a port access approach and the abdomen through a port access approach.

A variety of procedures may be performed with the systems and devices of the present invention. The following procedures are intended to provide suggestions for use and are by no means considered to limit such usage: laryngoscopy, rhinoscopy, pharyngoscopy, bronchoscopy, sigmoidoscopy, colonoscopy, esophagogastroduodenoscopy (EGD) which enables the physician to look inside the esophagus, stomach, and duodenum.

In addition, endoscopic retrograde cholangiopancreatography (ERCP) may be achieved which enables the surgeon to diagnose disease in the liver, gallbladder, bile ducts, and pancreas. In combination with this process endoscopic sphincterotomy can be done for facilitating ductal stone removal. ERCP may be important for identification of abnormalities in the pancreatic and biliary ductal system. Other treatments include cholecystectomy (removal of diseased gallbladder), CBD exploration (for common bile duct stones), appendicectomy (removal of diseased appendix), hernia repair TAP, TEPP and other (all kinds of hernia), fundoplication and HISS procedures (for gastro esophageal reflux disease), repair of duodenal perforation, gastrostomy for palliative management of late stage upper G.I.T. carcinoma), selective vagotomy (for peptic ulcer disease), splenectomy (removal of diseased spleen), upper and lower G.I. endoscopies (diagnostic as well as therapeutic endoscopies), pyloroplastic procedures (for children's congenital deformities), colostomy, colectomy, adrenalectomy (removal of adrenal gland for pheochromocytoma), liver biopsy, gastrojejunostomy, subtotal liver resection, gastrectomy, small intestine partial resections (for infarction or stenosis or obstruction), adhesions removal, treatment of rectum prolaps, Heller's Myotomy, devascularization in portal hypertension, attaching a device to a tissue wall and local drug delivery to name a few.

As mentioned previously, elongate body 120 has a proximal end 122 and a distal end 124 terminating in a distal tip 126. Elongate body 120 may include one or more sections or portions of elongate body 120 in which each section may be configured to bend or articulate in a controlled manner. A first section along elongate body 120 may be adapted to be deflectable and/or steerable, shape-lockable, etc. A second section, which may be located distally of and optionally adjacent to the first section along elongate body 120, may be adapted to retroflex independent of in conjunction with the first section. In one variation, this second section may be laterally stabilized and deflectable in a single plane. An optional third section, which may be located distally of and optionally adjacent to the second section, may be adapted to be a steerable portion, e.g., steerable within any axial plane in a 360-degree circumference around the shaft.

When a third section is utilized as the most distal section along elongate body 120, such steerability may allow for movement of the distal tip of elongate body 120 in a variety of directions. Such sections will be further described below. It may be appreciated that the elongate body 120 may be comprised of any combination of sections and may include such sections in any arrangement. Likewise, the elongate body 120 may be comprised of any subset of the three sections, e.g., first section and third section, or simply a third section. Further, additional sections may be present other than the three sections described above. Furthermore, multiple sections of a given variety, e.g. multiple sections adapted to be articulated as second section above, may be provided. Finally, one or all three sections may be independently lockable, as will be described below.

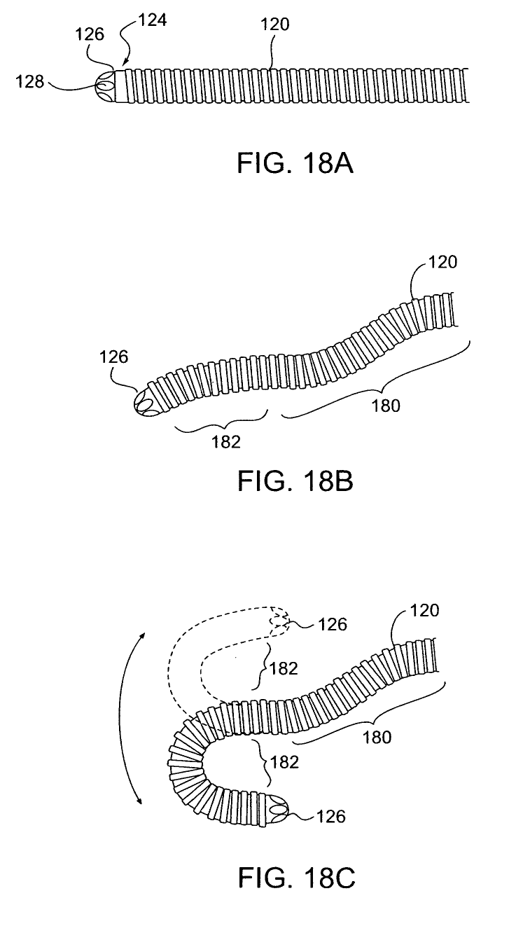

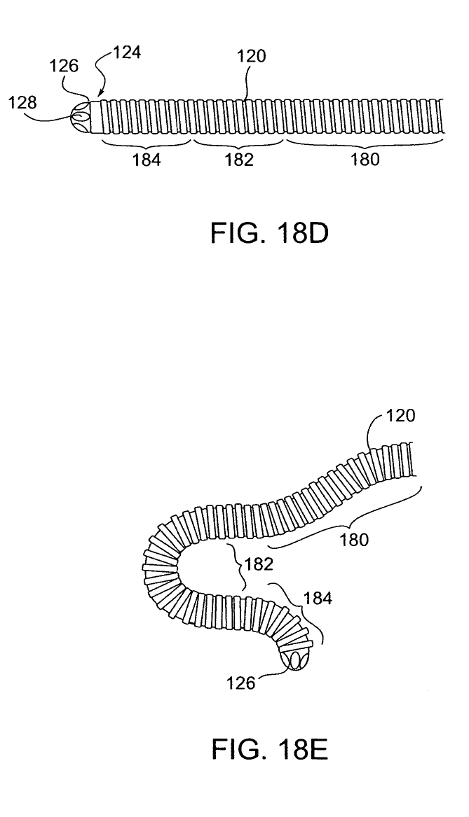

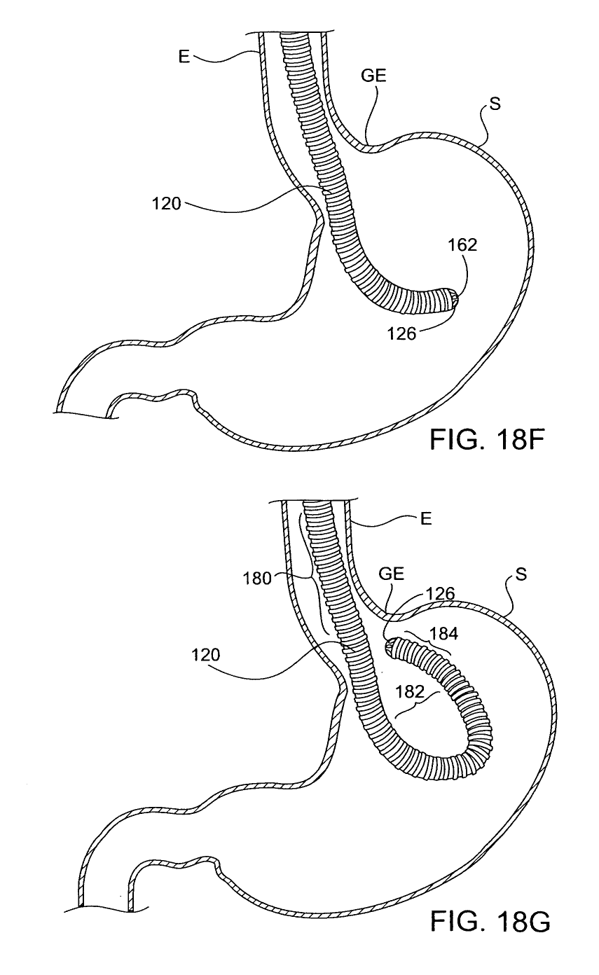

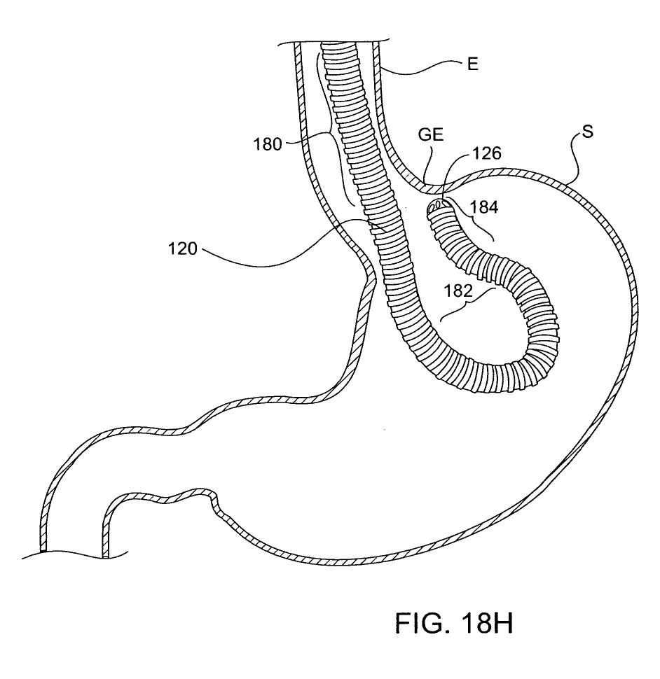

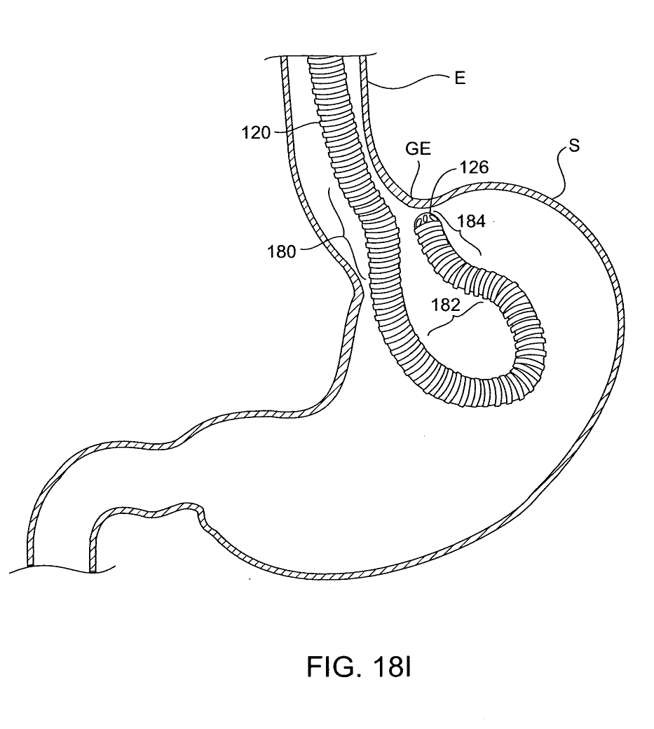

One variation of the elongate body 120 is illustrated in FIG. 18A in a straightened configuration. Only elongate body 120 is shown in these illustrations and the end effector assembly with off-axis tool arms and off-axis visualization has been omitted merely for the sake of clarity. Because the elongate body 120 is used to access an internal target location within a patient's body, elongate body 120 may include a deflectable and/or steerable shaft 120. Thus, FIG. 18B illustrates the elongate body 120 having various curvatures in its deflected or steered state. The elongate body 120 may be steerable so that the elongate body 120 may be advanced through unsupported anatomy and directed to desired locations within hollow body cavities. In this example, the elongate body 120 includes a first section 180 which is proximal to a second section 182, as indicated in FIG. 18B. Although both sections 180, 182 may be steerable, first section 180 may be adapted to lock its configuration while the second section 182 is further articulatable, as illustrated in FIG. 18C where first section 180 is shown in a locked position and the second section 182 is shown in various retroflexed positions.

When retroflexed, second section 182 may be curved or curled laterally outwardly so that the distal tip 126 is directable toward the proximal end 122 of the elongate body 120. Moreover, the second section 182 may be configured to form an arc which traverses approximately 270 degrees, if so desired. Optionally, the second section 182 also may be locked, either when retroflexed or in any other position. As should be understood, first section 180 optionally may not be steerable or lockable. For example, section 180 may comprise a passive tube extrusion.

A further variation of elongate body 120 is illustrated in FIG. 18D, in a straight configuration, and in FIG. 18E, in a deflected or steered state having various curvatures. In this variation, elongate body 120 may include a first section 180 proximal to a second section 182, which is proximal to a third section 184. First section 180 may be flexible or semi-flexible, e.g. such that the section 180 is primarily moveable through supported anatomy, or is moveable through unsupported anatomy via one or more stiffening members disposed within or about the section. The first section 180 may be comprised of links or nestable elements which may enable the first section 180 to alternate between a flexible state and a rigidized stated.

Optionally, first section 180 may comprise locking features for locking the section in place while the second section 182 is further articulated. Typically, the second section 182 may be configured to be adapted for retroflexion. In retroflexion, as illustrated in FIG. 18E, second section 182 may be curved or curled laterally and outwardly so that a portion of second section 182 is directed toward the proximal end 122 of the elongate body 120. It may be appreciated that second section 182 may be retroflexed in any desired direction. Optionally, second section 182 may also be locked, either in retroflexion or in any other position.

Further, first section 180 and second section 182 may be locked in place while third section 184 is further articulated. Such articulation is typically achieved by steering, such as with the use of pullwires. The distal tip 126 preferably may be steered in any direction relative to second section 182. For example, with second section 182 defining an axis, third section 184 may move within an axial plane, such as in a wagging motion. The third section 184 may move through any axial plane in a 360 degree circumference around the axis; thus, third section 184 may be articulated to wag in any direction. Further, third section 184 may be further steerable to direct the distal tip 126 within any plane perpendicular to any of the axial planes. Thus, rather than wagging, the distal tip 126 may be moved in a radial manner, such as to form a circle around the axis. FIG. 18E illustrates third section 184 steered into an articulated position within an axial plane.

The variation of elongate body 120 illustrated in FIGS. 18D and 18E having three sections 180, 182, 184 with varying movement capabilities are shown in FIGS. 18F and 18H in an example of positioning elongate body 120 within a stomach S through an esophagus E. Since elongate body 120 may be deflectable and at least some of the sections 180, 182, 184 may be steerable, elongate body 120 may be advanced through the tortuous or unpredictably supported anatomy of the esophagus and into the stomach S while reducing a risk of distending or injuring the organs, as shown in FIG. 18F. Once the distal tip 126 has entered the stomach, second section 182 may be retroflexed as illustrated in FIG. 18G. During retroflexion, distal tip 126 may traverse an arc having a continuous radius of curvature, e.g., approximately 270 degrees with a radius of curvature between about 5 to 10 cm. By retroflexing, distal tip 126 may be directed back towards first section 180 near and inferior to gastroesophageal junction GE. Second section 182 may be actively retroflexed, e.g. via pullwires, or it may be passively retroflexed by deflecting the section off a wall of stomach S while advancing elongate body 120.

Second section 182 may be configured to be shape-lockable in the retroflexed configuration. The distal tip 126 may then be further articulated and directed to a specific target location within the stomach. For example, as shown in FIG. 18H, the distal tip 126 may be steered toward a particular portion of the gastroesophageal junction GE. Third section 184 may optionally be shape-locked in this configuration. Off-axis tools and off-axis visualization may then be deployed through or from elongate body 120, as described above, to perform any number of procedures.

FIG. 18I shows yet another example in which elongate body 120 may be articulated in a manner similar as shown above in FIG. 18H. In this variation, elongate body may comprise a first section 180 which is configured to bend or curve in any number of directions. One particular variation may configure first section 180 to articulate in a direction opposite to a direction in which second section 182 bends. This opposed articulation may result in an elongate body 120 which conforms into a question-mark shape to facilitate positioning of third section 184 within stomach S, particularly for procedures which may be performed near or at the gastroesophageal junction GE. First section 180 may be configured to automatically conform into its opposed configuration upon rigidizing elongate body 120 or it may alternatively be articulated into its configuration by the physician.

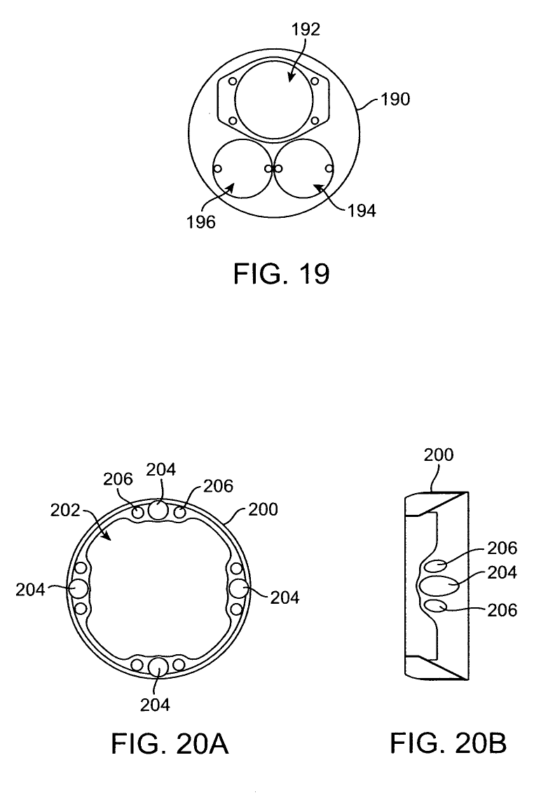

Turning now to the construction of the individual links which may form elongate body, FIGS. 19, 20A, and 20B show examples of link variations which may be utilized. FIGS. 20A and 20B show end and side views, respectively, of one variation of a link which may be utilized for construction of elongate body 120. An exemplary elongate body link 200 may be comprised generally of an open lumen 202 through any number of separate lumens, e.g., tool arm lumens, visualization lumens, etc., may be routed through.

The periphery defining open lumen 202 may define a number of openings for passage of various control wires, cables, optical fibers, etc. For instance, control wire lumens 204 may be formed at uniform intervals around the link 200, e.g., in this example, there are four control wire lumens 204 shown uniformly positioned about the link 200, although any number of lumens may be utilized as practicable and depending upon the desired articulation of elongate body 120. Elongate body link 200 may also comprise a number of auxiliary control lumens 206 spaced around body link 200 and adjacent to control wire lumens 204. Any number of biocompatible materials may be utilized in the construction of links 200, e.g., titanium, stainless steel, etc.

Aside from the elongate body links 200, one variation for a terminal link 190 may be seen in FIG. 19. Terminal link 190 may be utilized as an interface link between elongate body 120 and the distal end effector assembly 12. In the variation shown in FIG. 19, three lumens are utilized in terminal link 190 for a visualization lumen 192 and two tool arm channels 194, 196. In other variations for the terminal link, additional lumens may be defined through the link. In the case of an end effector having tools and a visualization lumen attached or coupled directly to the distal end of elongate body 120, the off-axis tools arms and off-axis articulatable lumen may be connected directly to terminal link 190.

Further examples and details of link construction may be seen in further detail in U.S. Pat. Pubs. 2004/0138525 A1; 2004/0138529 A1; 2004/0249367 A1; and 2005/0065397 A1, each of which has been incorporated above herein by reference

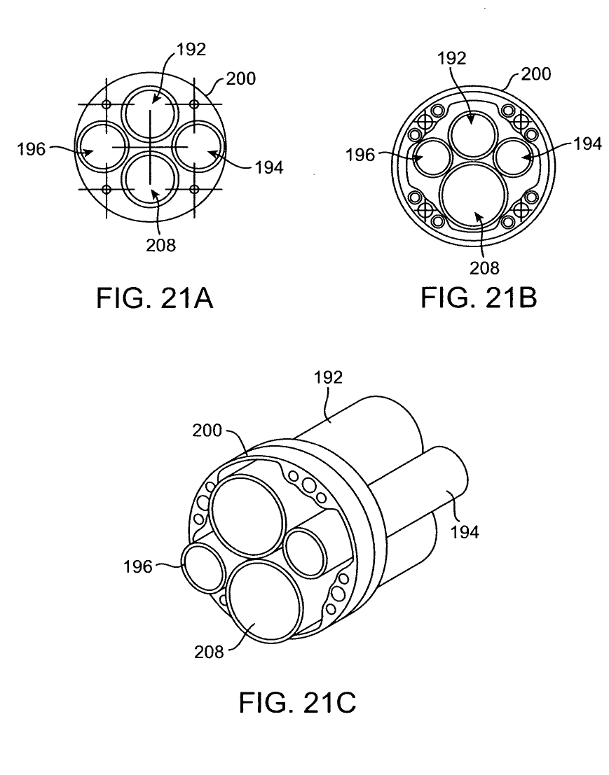

Arrangement of the individual lumens routed through elongate body 120 may be accomplished in any number of ways. For example, FIGS. 21A and 21B show end views of possible lumen arrangements where four lumens are utilized through elongate body 120. The variation in FIG. 21A shows elongate body link 200 where visualization lumen 192 and auxiliary instrument lumen 208 may be of a similar size diameter. Lumens 192, 208 may be positioned adjacently to one another with tool arm channels 194, 196 positioned on either side of lumens 192, 208.

In another variation, auxiliary instrument lumen 208 may be adjacently positioned and larger than visualization lumen 192, in which case tool arm channels 194, 196 may be positioned on either side of visualization lumen 192. In the spaces or interstices through link 200 between the visualization lumen 192, auxiliary instrument lumen 208, or either tool arm channels 194, 196, multiple smaller diameter lumens may be routed through for any number of additional features, e.g., insufflation, suction, fluid delivery, etc. FIG. 21C shows a perspective view of a single elongate body link 200 with visualization lumen 192, auxiliary instrument lumen 200, and tool arm channels 194, 196 routed therethrough.

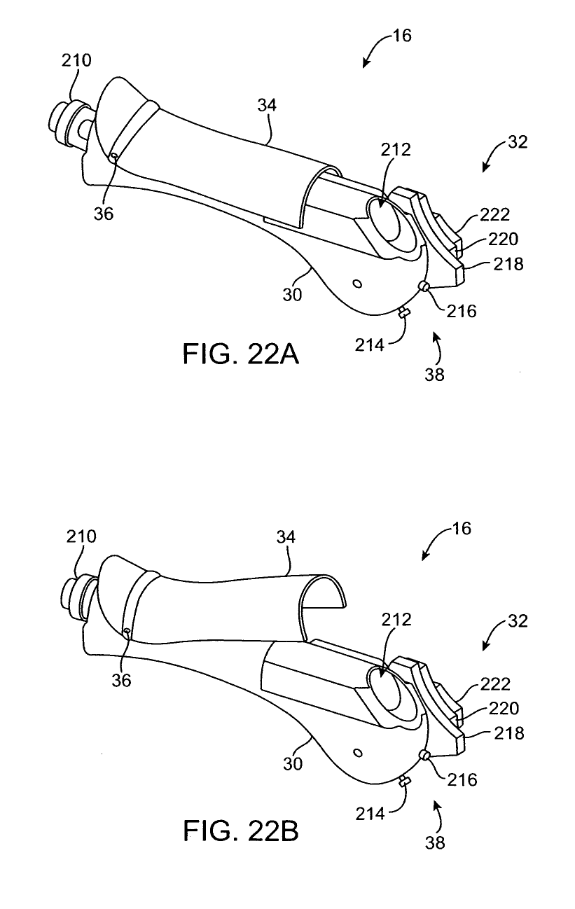

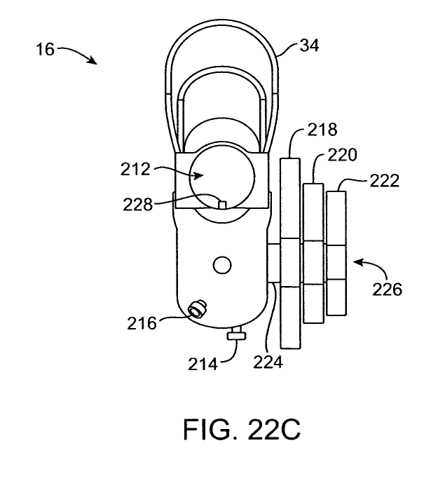

Turning now to the handle for endoluminal assembly 10, one variation of handle assembly may be seen in the perspective views of FIGS. 22A and 22B. Handle assembly 16 may generally comprise, in one variation, handle 30 which is connectable to the proximal end of elongate body 120 via elongate body interface 210. Coupling between the elongate body 120 and interface 210 may be accomplished in a number of different ways, e.g., interference fit, detents, etc., or the proximal link of elongate body 120 and interface 210 may be held adjacently to one another by routing control wires from handle 30 through interface 210 and into elongate body 120.

Interface 210 may also be adapted to travel proximally or distally relative to handle 30 when rigidizing control 34 is actuated about pivot 36 to actuate a rigidized or shape-locked configuration in elongate body 120. An example is shown in FIG. 22A where control 34 is depressed against handle 30 to advance interface 210 distally from handle 30. This distal movement of interface 210 compresses the links throughout elongate body 120 to rigidize its configuration. Likewise, as shown in FIG. 22B, when control 34 is released or pivoted away from handle 30, interface 210 may be configured to travel proximally relative to handle 30 such that a connected elongate body 120 is released into a flexible state by decompression of its links. Further details of mechanisms and methods for link compression for actuating a rigid shape of elongate body 120 may be seen further detail in U.S. Pat. Nos. 6,783,491; 6,790,173; and 6,837,847, each of which has been incorporated by reference above.

Handle 30 may also define an elongate body entry lumen 212 which may be defined near or at a proximal end of handle 30. Entry lumen 212 may define one or more openings for the passage of any of the tools and instruments, as described herein, through handle 30 and into elongate body 120. One or more ports, e.g., ports 214, 216, which are in fluid communication with one or more lumens routed through elongate body 120, as described above, may also be positioned on handle 30 and used for various purposes, e.g., insufflation, suction, irrigation, etc.

Additionally, handle 30 may further include a number of articulation or manipulation controls 32 for controlling elongate body 120 and/or end effector assembly 12. As shown in FIGS. 22A and 22B, control assembly 32 in this variation may include a first control 218 for manipulating or articulating first section 180; a second control 220 for manipulating or articulating second section 182 in a first plane; and a third control 222 for manipulating or articulating second section 182 in a second plane. In this variation of handle assembly 16, control assembly 32 is configured to have several control wheels which are adjacently positioned relative to one another over a common control axis 224, as shown in the end view of handle assembly 16 in FIG. 22C. Control assembly 32 may also include a locking mechanism 226 which may be configured to lock each of the controls 218, 220, 222 individually or simultaneously to lock a configuration of each section.

Moreover, each of the controls 218, 220, 222 may be configured to articulate their respective sections along elongate body 120 even when rigidizing control 34 has been articulated to rigidize a shape of the elongate body 120. In alternative variations, handle assembly 16 may include additional controls for additional sections of elongate body 120. Moreover, alternative configurations for the control assembly 32 may also include articulating levers or sliding mechanisms along handle 30 as control wheels are intended to be merely illustrative of the type of control mechanisms which may be utilized.

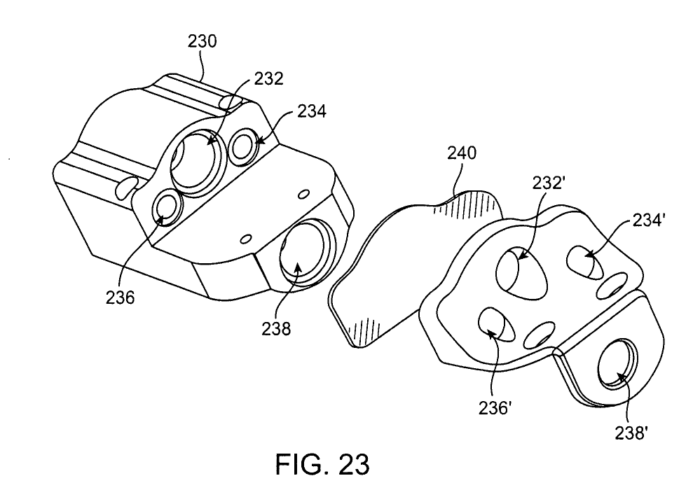

As mentioned above, entry lumen 212 may define one or more openings for the passage of any of the tools and instruments, as described herein, through handle 30 and into elongate body 120. To manage the insertion and sealing of multiple lumens routed through handle assembly 16 and elongate body 120, a port assembly may be connected or attached to handle 30 proximally of entry lumen 212 in a fluid-tight seal. A port assembly alignment post 228 for aligning such a port assembly may be seen in the end view of FIG. 22C. An example of such a port assembly 230 is shown in the perspective view of FIG. 23. Port assembly 230 may be seen having a visualization port lumen 232 for the insertion and passage of a visualization tool, as well as tool ports 234, 236 on either side of visualization port lumen 232 for the insertion of tools, as described above. Auxiliary instrument port 238 may also be seen on port assembly 230.

To maintain a fluid-tight seal through handle assembly 16 and elongate body 120 during instrument insertion, movement, and withdrawal in the patient body, a removable gasket 240 made from a compliant material, e.g., polyurethane, rubber, silicon, etc., may be positioned between ports 232, 234, 236, 238 of port assembly 230 and a retainer for securely retaining the gasket against assembly 230. The retainer may also have ports 232′, 234′, 236′, 238′ defined therethrough for alignment with their respective ports in assembly 230 for passage of the tools or instruments.

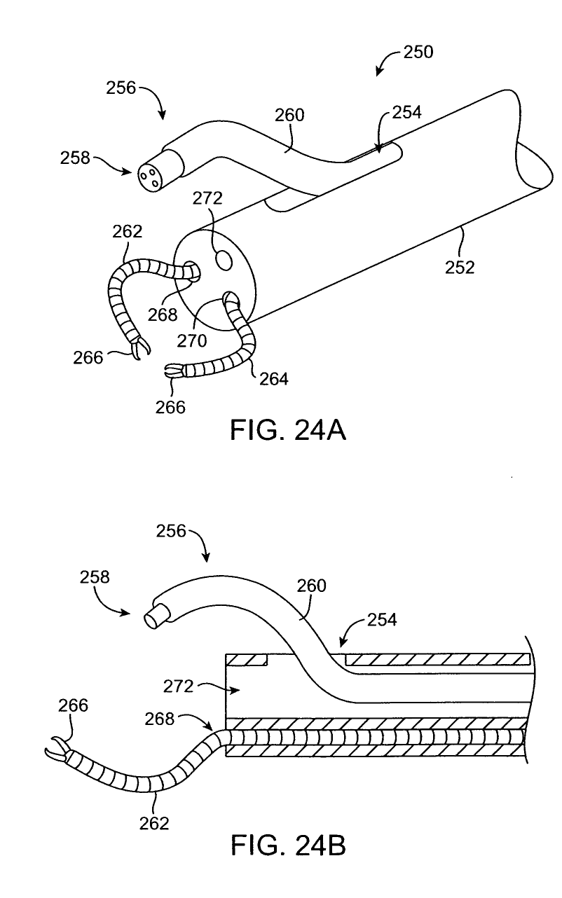

Other configurations for the end effector assembly may also be made utilizing a number of variations. FIGS. 24A and 24B show perspective and partial cross-sectional views, respectively, of a variation of end effector assembly 250. As illustrated, elongate body 252 may be a shape-lockable or rigidizable body which may be steerable or non-steerable, as described above, or it may generally be a passively flexible body which may be steerable or non-steerable as well. In either case, an opening 254 may be defined through an outer surface near or at a distal end of elongate body 252.

A visualization assembly 256, which may generally comprise an endoscope 258 having a bendable or flexible section 260 near or at its distal end, may be advanced through an endoscope or auxiliary instrument lumen 272 defined through elongate body 252 and advanced through opening 254. Endoscope 258 may be advanced through opening 254 such that its flexible section 260 enables the end of endoscope 258 to be positioned in an off-axis configuration distal of elongate body 252. Alternatively, endoscope 258 may be advanced entirely through lumen 272 such that it is disposed at the distal end of lumen 272 or projects distally therefrom to provide visualization of the tissue region of interest. First and second articulatable tool arms 262, 264 having one or more tools 266 upon their respective distal ends, as described above, may also be advanced through respective first and second tool lumens 268, 270. Tool arms 262, 264 may be disposed distally of elongate body 252 such that they are within the visualization field provided by the off-axis endoscope 258.

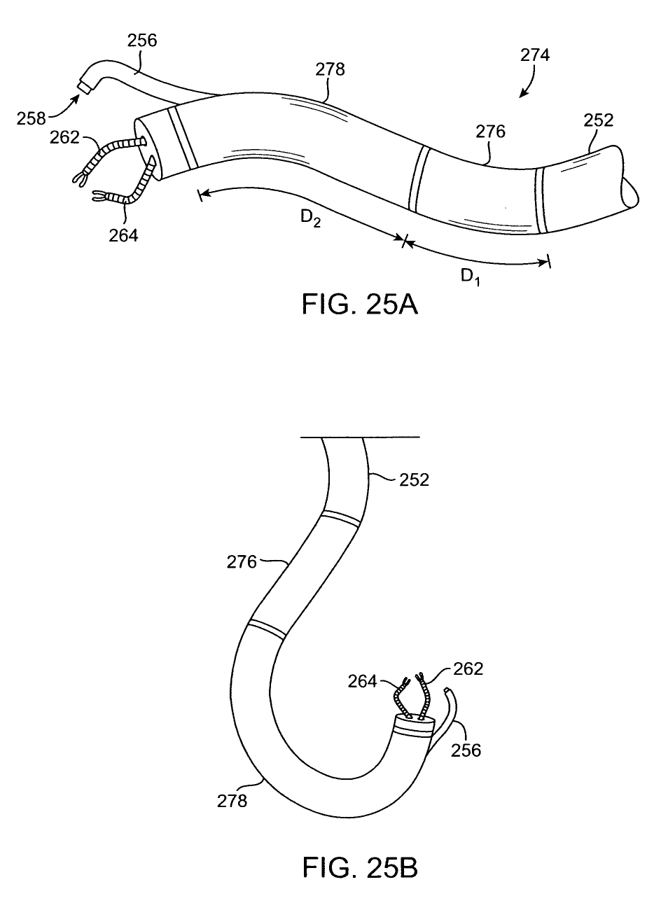

In another variation as shown in FIGS. 25A and 25B, elongate body 274 may comprise bendable or articulatable sections of varying lengths. Elongate body 274 in this variation may be shape-lockable or rigidizable along its length, as above, or it may have a passively flexible length. For example, elongate body 252 may have a first section 276 having a length D1 and a second section 278 having a length D2 located distally of first section 276. In the example shown, the length D1 of first section 276 may be shorter than the length D2 of second section 278, although the length of D1 may be longer than D2 in another alternative. Moreover, in yet another alternative, the lengths D1 and D2 may be equal. In the variation shown, having a length of D1 shorter than length D2 may allow for the end effector assembly to be articulated into a variety of configurations, especially if first section 276 is articulated in a direction opposite to a direction in which second section 278 is articulated, as shown in FIG. 25B. Any of the end effector assemblies described herein may be utilized with elongate body 252 having various lengths of sections 276, 278.

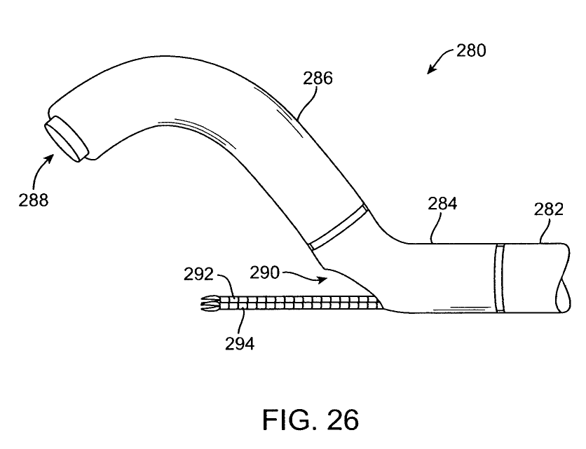

FIG. 26 shows a side profile of end effector assembly 280 in yet another variation. As shown, end effector assembly 280 may have an optionally shape-lockable elongate body 282 with articulatable first section 284 and second section 286. Second section 286 may be articulatable into an off-axis configuration such that an imager 288 positioned at its distal end may become positioned to view a region of interest accessible by first and second tool arms 292, 294, which may be passed through elongate body 282 and through opening 290 defined in first section 284 into the field-of-view provided by off-axis imager 288. Tool arms 292, 294 may be articulatable tool arms, as described above, or they may comprise any manner of conventional in-line tools.

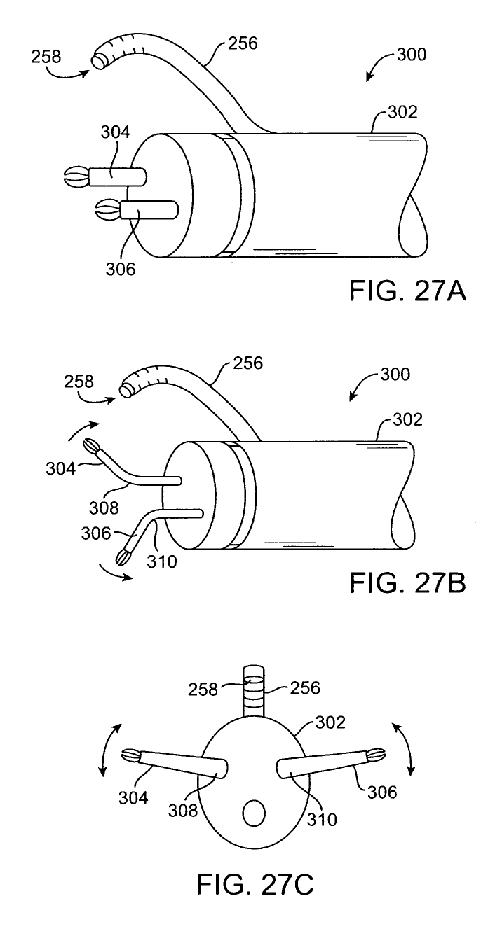

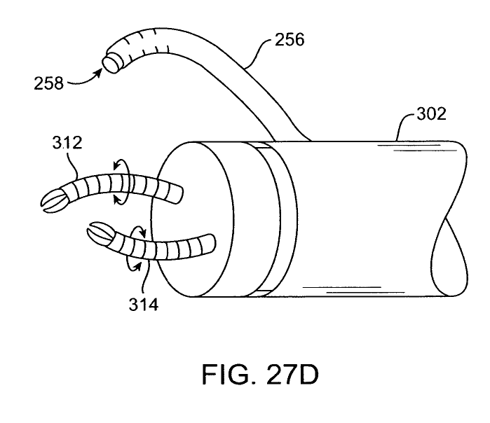

In yet another variation, FIGS. 27A and 27B show perspective views of end effector assembly 300 which may optionally comprise a shape-lockable elongate body 302 with off-axis visualization assembly 256, as above. In this variation, first and second tool arms 304, 306, respectively, may comprise arm members each having a first and second preset bending portion 308, 310, respectively, each configured to bend at a preset angle once free from the constraints of the tool lumens, as shown in FIG. 27B. Once unconstrained, tools arms 304, 306 may be rotated about its longitudinal axis, as shown in FIG. 27C, to accomplish any number of procedures on the tissue while visualized via off-axis endoscope 258. Tool arms 304, 306 may be fabricated from shape memory alloys, such as a Nickel-Titanium alloy, or from spring stainless steels, or any other suitable material which may allow for the tools arms 304, 306 to reconfigure itself from a first low-profile configuration to an off-axis deployment configuration.

FIG. 27D shows a perspective view of yet another variation in which elongate body 302 may have first and second articulatable tool arms 312, 314 which are freely rotatable about their respective longitudinal axes. Visualization assembly 256 may comprise any of the variations described above, particularly the variation as described for FIGS. 24A and 24B.

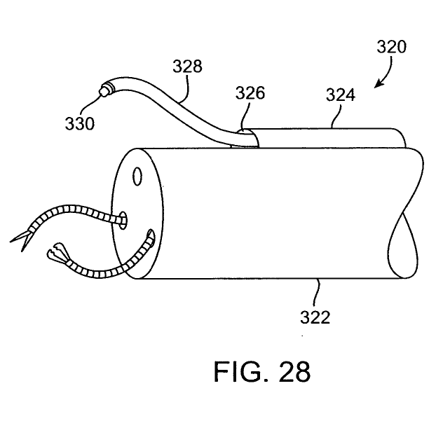

FIG. 28 shows a perspective view of another variation of end effector assembly 320 in which optionally shape-lockable elongate body 322 may comprise a separate visualization lumen 324 having a lumen opening 326 through which a guidewire 328 having a preset configuration may be advanced. Visualization lumen 324 may be integrated with elongate body 322 or separately attached to an outer surface of elongate body 322. Guidewire 328 may be comprised of a shape memory alloy, as above, and carry an imaging chip 330, e.g., a CCD imager, on a distal end of the guidewire 328. Guidewire 328 may be preset to reconfigure itself into an off-axis configuration to provide the off-axis visualization distally of elongate body 322, as shown. Furthermore, imaging chip 330 may be connected via wires through guidewire 328 to a monitor at a location proximal to elongate body 322 or imaging chip 330 may be adapted to wirelessly transmit images to a receiving unit external to a patient body. Moreover, guidewire 328 may also be advanced through a working lumen of elongate body 322 if so desired.

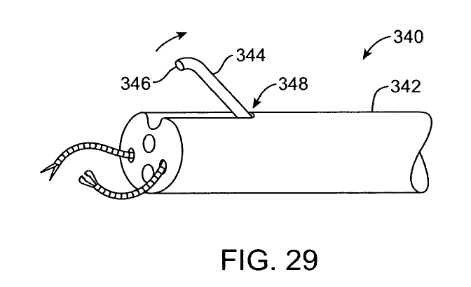

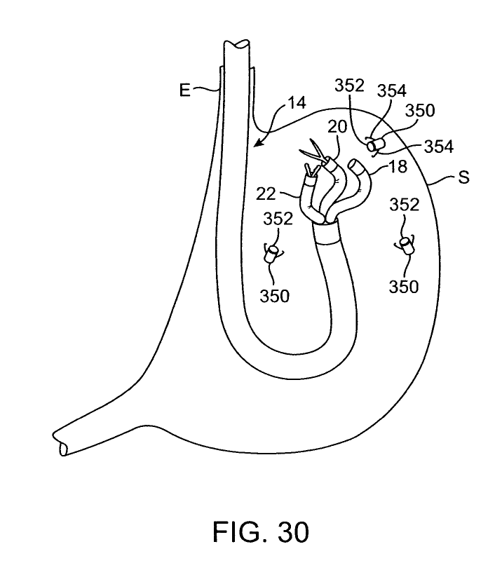

In another alternative, end effector assembly 340 shown in FIG. 29 may comprise an optionally shape-lockable body 342 having visualization member 344 pivotably mounted near or at a distal end of body 342 via pivot 348. Visualization member 344 may have an imager 346, e.g., an imaging chip such as a CCD chip, positioned upon a distal end of member 344, which may be configured to articulate about pivot 348 such that imager 346 is provided an off-axis view of the region distal of elongate body 342.

In another variation, the off-axis visualization may be provided, e.g., within the stomach S, via one or more capsules 350 having integrated imagers 352 positioned within one or more regions of the stomach S. Rather than, or in combination with, off-axis visualization lumen or platform 18, a number of imaging capsules 350 may be temporarily adhered to the interior stomach wall, e.g., via clips 354 attached to the capsule body. The imaging portions 352 of the capsules 350 may be positioned against the stomach wall such that one or more capsules 350 are pointed towards a tissue region of interest. The endoluminal assembly 10 may then be articulated towards the tissue region of interest with either off-axis visualization platform 18 or one or more capsules 350 providing a number of off-axis views for any number of procedures to be accomplished. Imaging capsules such as the PillCam™ are generally used for capsule endoscopy and may be commercially obtained from companies like Given Imaging Ltd. (Israel).

Although various illustrative embodiments are described above, it will be evident to one skilled in the art that a variety of combinations of aspects of different variations, changes, and modifications are within the scope of the invention. It is intended in the appended claims to cover all such changes and modifications that fall within the true spirit and scope of the invention.

1. An apparatus for tissue manipulation, comprising:
an elongate body defining a longitudinal axis and adapted for endoluminal advancement in a patient body;
at least one articulatable platform disposed near or at a distal region of the elongate body, the at least one articulating platform being adapted to articulate off-axis relative to the longitudinal axis; and
at least one articulatable member disposed near or at the distal region of the elongate body, the at least one articulatable member being adapted to articulate off-axis and manipulate a tissue region of interest.

2. The apparatus of claim 1 wherein the elongate body is adapted to selectively transition from a flexible state to a rigid state.

3. The apparatus of claim 1 wherein the elongate body further comprises a first articulatable section of the elongate body near or at the distal region, wherein the first articulatable section is adapted to bend via manipulation by a user.

4. The apparatus of claim 3 further comprising a second articulatable section of the elongate body located distal to the first articulatable section, wherein the second articulatable section is adapted to bend via manipulation by the user.

5. The apparatus of claim 4 wherein the first and second articulatable sections are manipulatable independently of one another.

6. The apparatus of claim 4 wherein the first articulatable section is adapted to articulate within a single plane relative to the elongate body.

7. The apparatus of claim 4 wherein the second articulatable section is adapted to have 4-way articulation relative to the first articulatable section.

8. The apparatus of claim 4 further comprising a third articulatable section of the elongate body located distal to the second articulatable section, wherein the third articulatable section is adapted to bend via manipulation by the user.

9. The apparatus of claim 4 wherein the first and second articulatable sections are each adapted to selectively transition from a flexible state to a rigid state.

10. The apparatus of claim 1 wherein the elongate body is comprised of a plurality of nested links rotatingly aligned serially with one another.

11. The apparatus of claim 1 wherein the elongate body defines a visualization lumen for passage of a visualization instrument therethrough.

12. The apparatus of claim 11 wherein the visualization lumen passes through the elongate body and through the at least one articulatable platform.

13. The apparatus of claim 1 wherein the elongate body defines at least two tool lumens for passage of tools therethrough.

14. The apparatus of claim 13 wherein each of the tool lumens passes through the elongate body and through at least two corresponding articulatable members disposed near or at the distal region of the elongate body.

15. The apparatus of claim 1 wherein the at least one articulatable platform comprises a platform pivotably coupled near or at the distal region of the elongate body such that the platform is movable from a first low-profile position to a second extended position.

16. The apparatus of claim 1 wherein the at least one articulatable platform comprises an elongate lumen which is adapted to articulate from a first low-profile position to a second off-axis position such that a distal end of the elongate lumen is directed to a region distal of the elongate body.

17. The apparatus of claim 16 wherein the elongate lumen further comprises an imager disposed at the distal end of the elongate lumen.

18. The apparatus of claim 17 wherein the imager comprises an endoscope.

19. The apparatus of claim 17 wherein the imager comprises an imaging chip.

20. The apparatus of claim 19 wherein the imaging chip is positioned on or along the elongate lumen such that the imaging chip images a field-of-view distal to the elongate body and off-axis relative to the longitudinal axis.

21. The apparatus of claim 19 further comprising a receiving unit located external to the patient body for wirelessly receiving visual images from the imaging chip.

22. The apparatus of claim 1 wherein the at least one articulatable platform comprises a guidewire adapted to reconfigure from a low-profile configuration to an off-axis configuration relative to the elongate body.

23. The apparatus of claim 1 wherein the at least one articulatable platform is adapted to be advanced distally through an opening defined along the elongate body and configured into an off-axis configuration.

24. The apparatus of claim 1 wherein the at least one articulatable element comprises an articulatable lumen adapted to position a distal tip of the articulatable lumen off-axis relative to the longitudinal axis.

25. The apparatus of claim 1 wherein the at least one articulatable member comprises at least one articulatable arm member.

26. The apparatus of claim 1 further comprising a tool positionable through the at least one articulatable member.

27. The apparatus of claim 1 further comprising a tool disposed upon a distal end of the at least one articulatable member.

28. The apparatus of claim 1 further comprising a second articulatable member disposed near or at the distal region of the elongate body, wherein the second articulatable member is adapted to articulate off-axis.

29. The apparatus of claim 28 wherein each of the off-axis articulatable members and the articulatable platform are adapted to provide for triangulation of the articulatable members when visualized from the articulatable platform.

30. The apparatus of claim 1 wherein the at least one articulatable member is adapted to rotate about a longitudinal axis of the articulatable member.

31. The apparatus of claim 1 wherein the at least one articulatable member is adapted to reconfigure from a low profile configuration into a preset off-axis configuration when advanced distally from the elongate body.

32. The apparatus of claim 1 further comprising an endoscope disposable within or along the at least one articulatable platform.

33. The apparatus of claim 1 further comprising a handle assembly coupled to a proximal end of the elongate body for controlling the apparatus.

34. A system for tissue manipulation, comprising:
an elongate body defining a longitudinal axis and adapted for endoluminal advancement in a patient body;
an elongate lumen which is adapted to articulate from a first low-profile position to a second position off-axis relative to the longitudinal axis such that a distal end of the elongate lumen is directed to a region distal of the elongate body; and
a first and a second articulatable arm member each disposed near or at the distal region of the elongate body, each articulatable member being adapted to articulate off-axis and manipulate a tissue region of interest.

35. The system of claim 34 wherein the elongate body is adapted to selectively transition from a flexible state to a rigid state.

36. The system of claim 34 wherein the elongate body further comprises a first articulatable section of the elongate body near or at the distal region, wherein the first articulatable section is adapted to bend via manipulation by a user.

37. The system of claim 36 further comprising a second articulatable section of the elongate body located distal to the first articulatable section, wherein the second articulatable section is adapted to bend via manipulation by the user.

38. The system of claim 37 further comprising a third articulatable section of the elongate body located distal to the second articulatable section, wherein the third articulatable section is adapted to bend via manipulation by the user.

39. The system of claim 34 wherein the elongate body defines a visualization lumen for passage of a visualization instrument therethrough.

40. The system of claim 39 wherein the visualization lumen passes through the elongate body and through the elongate lumen.

41. The system of claim 34 wherein the elongate body defines a first and a second tool lumen for passage of tools therethrough.

42. The system of claim 41 wherein each of the first and second tool lumens passes through the elongate body and through respective first and the second articulatable arm members.

43. The system of claim 34 wherein the elongate lumen further comprises an imager disposed at the distal end of the elongate lumen.

44. The system of claim 43 wherein the imager comprises an endoscope.

45. The system of claim 41 wherein the imager comprises an imaging chip.

46. The system of claim 34 further comprising a first tool positionable through the first articulatable arm member and a second tool positionable through the second articulatable arm member.

47. The system of claim 34 further comprising a first tool disposed upon a distal end of the first articulatable arm member and a second tool disposed upon a distal end of the second articulatable arm member.

48. A method for endoluminally performing a procedure upon a tissue region of interest, comprising:
endoluminally advancing an elongate body defining a longitudinal axis and adapted for advancement into a patient body;
articulating at least one platform disposed near or at a distal region of the elongate body into an off-axis configuration relative to the longitudinal axis, the at least one platform being adapted to provide visualization of a tissue region of interest distal to the elongate body; and
articulating at least one member disposed near or at the distal region of the elongate body into an off-axis configuration relative to the longitudinal axis, the at least one member being adapted to manipulate the tissue region of interest.

49. The method of claim 48 wherein endoluminally advancing comprises advancing the elongate body transesophageally into a stomach of the patient body.

50. The method of claim 48 wherein endoluminally advancing further comprises articulating a first articulatable section of the elongate body near or at a distal region of the elongate body.

51. The method of claim 50 further comprising articulating a second articulatable section of the elongate body located distal to the first articulatable section.

52. The method of claim 51 further comprising articulating a third articulatable section of the elongate body located distal to the second articulatable section.

53. The method of claim 48 wherein endoluminally advancing further comprises transitioning at least a portion of the elongate body from a flexible state to a rigid state.

54. The method of claim 48 wherein articulating at least one platform comprises reconfiguring an elongate lumen from a first low-profile position to a second position off-axis relative to the longitudinal axis.

55. The method of claim 54 further comprising visualizing the tissue region of interest via an imager positioned at a distal end of the elongate lumen.

56. The method of claim 55 wherein visualizing the tissue region comprises visualizing the tissue region via an endoscope positioned within the elongate lumen.

57. The method of claim 55 wherein visualizing the tissue region comprises visualizing the tissue region via an imaging chip positioned within the elongate lumen.

58. The method of claim 57 further comprising wirelessly transmitting visual images from the imaging chip to a receiving unit located externally to the patient body.

59. The method of claim 48 wherein articulating at least one member comprises articulating a first articulatable arm and a second articulatable arm each off-axis relative to the longitudinal axis.

60. The method of claim 59 further comprising manipulating the tissue region of interest with at least the first articulatable arm or the second articulatable arm.

61. The method of claim 59 further comprising manipulating a first tool disposed at a distal end of the first articulatable arm and manipulating a second tool disposed at a distal end of the second articulatable arm.
